SEQUENCE LISTING (36 sequences)

SEQ ID NO: 1 - protein, 9 aa, Artificial Sequence
  Synthetic polypeptide
GPLGVRGKC

SEQ ID NO: 2 - protein, 13 aa, Artificial Sequence
  Synthetic polypeptide
GGGSGRSANAKGC

SEQ ID NO: 3 - protein, 11 aa, Artificial Sequence
  Synthetic polypeptide
GSGSKIIGGGC

SEQ ID NO: 4 - protein, 12 aa, Artificial Sequence
  Synthetic polypeptide
GGLGPKGQTGGC

SEQ ID NO: 5 - protein, 13 aa, Artificial Sequence
  Synthetic polypeptide
  MISC_FEATURE (1)..(1): Modified by FAM
  MISC_FEATURE (11)..(11): Modified by CPQ2
  MISC_FEATURE (11)..(12): Modified by PEG2
GKPISLISSGKGC

SEQ ID NO: 6 - protein, 13 aa, Artificial Sequence
  Synthetic polypeptide
  MISC_FEATURE (1)..(1): Modified by FAM
  MISC_FEATURE (11)..(11): Modified by CPQ2
  MISC_FEATURE (11)..(12): Modified by PEG2
GKPISLISSGKGC

SEQ ID NO: 7 - protein, 25 aa, Artificial Sequence
  Synthetic polypeptide
  MISC_FEATURE (1)..(1): Modified by biotin
  MISC_FEATURE (15)..(16): Modified by DNP
  MISC_FEATURE (25)..(25): Modified by PEG
EGVNDNEEGFFSARKGPLGVRGKGC

SEQ ID NO: 8 - protein, 28 aa, Artificial Sequence
  Synthetic polypeptide
  MISC_FEATURE (1)..(1): Modified by biotin
  MISC_FEATURE (15)..(16): Modified by FAM
  MISC_FEATURE (28)..(28): Modified by PEG
EGVNDNEEGFFSARKGGGSGRSANAKGC

SEQ ID NO: 9 - protein, 26 aa, Artificial Sequence
  Synthetic polypeptide
  MISC_FEATURE (1)..(1): Modified by biotin
  MISC_FEATURE (15)..(16): Modified by TAMRA
  MISC_FEATURE (26)..(26): Modified by PEG
EGVNDNEEGFFSARKGSGSKIIGGGC

SEQ ID NO: 10 - protein, 27 aa, Artificial Sequence
  Synthetic polypeptide
  MISC_FEATURE (1)..(1): Modified by biotin
  MISC_FEATURE (15)..(16): Modified by Cy7
  MISC_FEATURE (27)..(27): Modified by PEG
EGVNDNEEGFFSARKGGLGPKGQTGGC

SEQ ID NO: 11 - protein, 24 aa, Artificial Sequence
  Synthetic polypeptide
  MISC_FEATURE (1)..(1): Modified by biotin
  MISC_FEATURE (15)..(16): Modified by AF488
EGVNDNEEGFFSARKGGLGGGAGC

SEQ ID NO: 12 - protein, 8 aa, Artificial Sequence
  Synthetic polypeptide
SLKRYGGG

SEQ ID NO: 13 - protein, 8 aa, Artificial Sequence
  Synthetic polypeptide
AAFRSRGA

SEQ ID NO: 14 - protein, 8 aa, Artificial Sequence
  Synthetic polypeptide
  misc_feature (1)..(2): Xaa can be any naturally occurring amino acid
  misc_feature (7)..(8): Xaa can be any naturally occurring amino acid
XXFRFFXX

SEQ ID NO: 15 - protein, 6 aa, Artificial Sequence
  Synthetic polypeptide
QSVGFA

SEQ ID NO: 16 - protein, 7 aa, Artificial Sequence
  Synthetic polypeptide
LGLEGAD

SEQ ID NO: 17 - protein, 4 aa, Artificial Sequence
  Synthetic polypeptide
GPLD

SEQ ID NO: 18 - protein, 6 aa, Artificial Sequence
  Synthetic polypeptide
LGVLIV

SEQ ID NO: 19 - protein, 6 aa, Artificial Sequence
  Synthetic polypeptide
GLVLVA

SEQ ID NO: 20 - protein, 6 aa, Artificial Sequence
  Synthetic polypeptide
PAALVG

SEQ ID NO: 21 - protein, 7 aa, Artificial Sequence
  Synthetic polypeptide
GPAGLAG

SEQ ID NO: 22 - protein, 14 aa, Artificial Sequence [sequence omitted]

SEQ ID NO: 23 - protein, 11 aa, Artificial Sequence
  Synthetic polypeptide
  MISC_FEATURE (1)..(1): Modified by 5FAM
  MISC_FEATURE (10)..(11): Modified by CPQ2-PEG2
GGPLGVRGKKC

SEQ ID NO: 24 - protein, 11 aa, Artificial Sequence
  Synthetic polypeptide
  MISC_FEATURE (1)..(1): Modified by QSY21
  MISC_FEATURE (10)..(11): Modified by Cy5-PEG2
GGPLGVRGKKC

SEQ ID NO: 25 - protein, 25 aa, Artificial Sequence
  Synthetic polypeptide
  MISC_FEATURE (1)..(1): Modified by biotin
  MISC_FEATURE (15)..(16): Modified by DNP
EGVNDNEEGFFSARKGPLGVRGKGC

SEQ ID NO: 26 - protein, 25 aa, Artificial Sequence
  Synthetic polypeptide
  MISC_FEATURE (1)..(1): Modified by biotin
  MISC_FEATURE (15)..(16): Modified by 5FAM
EGVNDNEEGFFSARKGPLGVRGKGC

SEQ ID NO: 27 - protein, 12 aa, Artificial Sequence
  Synthetic polypeptide
  MISC_FEATURE (1)..(1): Modified by 5FAM
  MISC_FEATURE (10)..(11): Modified by CPQ2-PEG2
GGLGPKGQTGKC

SEQ ID NO: 28 - protein, 27 aa, Artificial Sequence
  Synthetic polypeptide
  MISC_FEATURE (1)..(1): Modified by biotin
  MISC_FEATURE (15)..(16): Modified by Cy7
EGVNDNEEGFFSARKGGLGPKGQTGGC

SEQ ID NO: 29 - protein, 15 aa, Artificial Sequence
  Synthetic polypeptide
  MISC_FEATURE (1)..(1): Modified by 5FAM
  MISC_FEATURE (13)..(14): Modified by CPQ2-PEG2
GGGSGRSANAKGKGC

SEQ ID NO: 30 - protein, 28 aa, Artificial Sequence
  Synthetic polypeptide
  MISC_FEATURE (1)..(1): Modified by biotin
  MISC_FEATURE (15)..(16): Modified by 5FAM
EGVNDNEEGFFSARKGGGSGRSANAKGC

SEQ ID NO: 31 - protein, 10 aa, Artificial Sequence
  Synthetic polypeptide
  MISC_FEATURE (1)..(2): Modified by PEG2
CCRGDKGPDC

SEQ ID NO: 32 - protein, 7 aa, Artificial Sequence
  Synthetic Polypeptide
PLGVRGK

SEQ ID NO: 33 - protein, 7 aa, Artificial Sequence
  Synthetic Polypeptide
LGPKGQT

SEQ ID NO: 34 - protein, 8 aa, Artificial Sequence
  Synthetic Polypeptide
SGRSANAK

SEQ ID NO: 35 - protein, 6 aa, Artificial Sequence
  Synthetic Polypeptide
SGSKII

SEQ ID NO: 36 - protein, 9 aa, Artificial Sequence
  Synthetic Polypeptide
CRGDKGPDC

(19) United States
(12) Patent Application Publication
(10) Pub. No.: US 2020/0249194 A9
(43) Pub. Date: Aug. 6, 2020

(54) PROSTATE PROTEASE NANOSENSORS AND USES THEREOF

(71) Applicant: Massachusetts Institute of Technology, Cambridge, MA (US)

(72) Inventors: Jaideep S. Dudani, Boston, MA (US); Sangeeta N. Bhatia, Lexington, MA (US)

(73) Assignee: Massachusetts Institute of Technology, Cambridge, MA (US)

(21) Appl. No.: 16/159,340

(22) Filed: Oct. 12, 2018

Related U.S. Application Data
(60) Provisional application No. 62/571,644, filed on Oct. 12, 2017.

(51) Int. Cl. G01N 27/327; C12Q 1/00; G01N 33/574; G01N 33/543

(52) U.S. Cl. CPC G01N 27/3278; G01N 33/5438; G01N 33/57434; C12Q 1/005

(57) ABSTRACT

In some aspects, the disclosure relates to compositions and method for detection, classification, and treatment of prostate cancer. In some embodiments, the disclosure relates to prostate protease nanosensors comprising a scaffold linked to a prostate-specific substrate that include a detectable marker capable of being released from the prostate protease nanosensor when exposed to an enzyme present in a prostate. In some embodiments, the disclosure relates to methods of classifying prostate cancer in a subject based upon detection of detectable markers in a sample obtained from a subject who has been administered prostate protease nanosensors.

### CROSS-REFERENCE TO RELATED APPLICATIONS

This application claims the benefit of priority under 35 U.S.C. § 119(e) of U.S. provisional application Ser. No. 62/571,644, filed Oct. 12, 2017, the disclosure of which is incorporated by reference here in its entirety.

### GOVERNMENT SUPPORT

This invention was made with Government support under Grant Nos. P30 CA014051 and P30 ES002109 awarded by the National Institutes of Health. The Government has certain rights in the invention.

### BACKGROUND

Prostate cancer is the most common noncutaneous cancer in men, with a lifetime risk for a U.S. male of about 1 in 6. Mortality from prostate cancer, however, is low relative to its prevalence. This discrepancy has led to poor patient management, especially for patients with low-grade prostate cancer. The current standard of care, prostate specific antigen (PSA) screening, has poor predictive value and sensitivity. Currently, 85% of prostate cancer diagnoses occur when tumors are low or medium grade, but 30% percent of these patients harbor high-grade cancer that is underrepresented on their biopsies. Due to biomarkers with limited sensitivity, there are several unmet needs for prostate cancer.

### SUMMARY

In some aspects, the disclosure relates to methods and compositions for identification, classification and/or treatment of certain cancers, such as prostate cancers. The disclosure is based, in part, on synthetic biomarkers (e.g., protease nanosensors) that are capable of distinguishing (e.g., classifying) aggressive and indolent cancers (e.g., prostate cancers) by interrogating protease activity levels in a tumor microenvironment, such as the prostate.

Accordingly, in some aspects, the disclosure provides a prostate protease nanosensor comprising a scaffold linked to a prostate-specific substrate, wherein the prostate-specific substrate includes a detectable marker, whereby the detectable marker is capable of being released from the prostate protease nanosensor when exposed to an enzyme present in a prostate (e.g., a prostate cancer-associated enzyme).

In some aspects, the disclosure provides a composition comprising at least 2 (e.g., 2 to 50, 5 to 30, 2 to 20, more than 20, etc.) different prostate protease nanosensors, wherein the different prostate protease nanosensors comprise a different substrate (e.g., comprise different prostate-specific substrates).

In some embodiments, a composition comprises a multiplexed library of substrates (e.g., prostate cancer-specific substrates). In some embodiments, a multiplexed library of substrates comprises 2 or more (e.g., at least 2, 3, 4, 5, 10, 15, 20, or more) substrates. In some embodiments, a multiplexed library comprises between 2 and 30 (e.g., any integer between 2 and 30, inclusive) substrates.

In some aspects, the disclosure provides a method for classifying cancer in a subject, the method comprising detecting in a biological sample obtained from a subject that has been administered a prostate protease nanosensor or composition as described herein (e.g., composition containing one or more different prostate protease nanosensors), wherein the biological sample is not derived from the prostate of the subject, one or more detectable markers that have been released from one or more prostate protease nanosensors when exposed to an enzyme present in the prostate of the subject, and classifying the subject as having an indolent cancer or an aggressive cancer based on the identity of the detectable markers present in the biological sample, wherein the presence of the detectable markers in the biological sample is indicative of one or more cancer-associated enzymes being present in an active form within the prostate of the subject.

In some embodiments, a scaffold comprises a high molecular weight protein, a high molecular weight polymer, or a nanoparticle scaffold. In some embodiments, a scaffold is greater than about 40 kDa. In some embodiments, a scaffold comprises a multi-arm polyethylene glycol molecule (multi-arm PEG). In some embodiments, a multi-arm PEG comprises between 2 and 20 arms. In some embodiments, a multi-arm PEG comprises more than 20 arms (e.g., 30, 50, 100, 200, or more arms). In some embodiments, a multi-arm PEG has a total molecular weight greater than 40 kDa.

In some embodiments, a scaffold comprises an iron oxide nanoparticle (IONP). In some embodiments, an IONP is between about 10 nm and about 20 nm (e.g., any value between 10 nm and 20 nm, inclusive) in size, for example as measured by average particle diameter.

In some embodiments, a scaffold is linked to a single protease-specific substrate. In some embodiments, a scaffold is linked to 2 to 20 (e.g., any integer between 2 and 20, inclusive) different protease-specific substrates. In some embodiments, a scaffold is linked to 2 to 4 (e.g., 2, 3, or 4) different protease-specific substrates.

In some embodiments, a cancer substrate is cleaved by an enzyme associated with prostate cancer. In some embodiments, a cancer substrate is a substrate cleaved by an enzyme selected from MMP11, MMP13, KLK2, KLK3, KLK4, KLK5, KLK12, KLK14, PRSS3, uPA, MMP3, MMP26, HPN, MMP10, MMP9, ADAM12, or any combination thereof. In some embodiments, a substrate comprises the amino acid sequence set forth as GPLGVRGKC (SEQ ID NO: 1), PLGVRGK (SEQ ID NO: 32), LGPKGQT (SEQ ID NO: 33), SGRSANAK (SEQ ID NO: 34), SGSKII (SEQ ID NO: 35), GGGSGRSANAKGC (SEQ ID NO: 2), GSGSKIIGGGC (SEQ ID NO: 3), or GGLGPKGQTGGC (SEQ ID NO: 4).

In some embodiments, a cancer substrate is a metastatic cancer (e.g., aggressive cancer) substrate. In some embodiments, a metastatic cancer substrate is cleaved by one or more proteases selected from KLK2, KLK5, KLK12, KLK14, MMP3, ADAM12, MMP11, MMP13, PRSS3, and uPA. In some embodiments, a metastatic cancer substrate comprises the amino acid sequence set forth as GGGSGRSANAKGC (SEQ ID NO: 2), SGRSANAK (SEQ ID NO: 34), LGPKGQT (SEQ ID NO: 33), or GGLGPKGQTGGC (SEQ ID NO: 4). In some embodiments, a cancer substrate is a non-metastatic cancer (e.g., an indolent cancer) substrate. In some embodiments, a non-metastatic cancer substrate comprises the amino acid sequence set forth as GPLGVRGKC (SEQ ID NO: 1), PLGVRGK (SEQ ID NO: 32), SGSKII (SEQ ID NO: 35), or GSGSKIIGGGC (SEQ ID NO: 3).

In some embodiments, a detectable marker is a peptide, nucleic acid, small molecule, fluorophore (e.g., a fluorophore, or a fluorophore/quencher pair, such as a FRET pair), carbohydrate, particle, radiolabel, MRI-active compound, ligand encoded reporter, or isotope coded reporter molecule (iCORE).

In some aspects, the disclosure provides a method comprising detecting in a biological sample obtained from a subject that has been administered a prostate protease nanosensor or a composition as described by the disclosure one or more detectable markers that have been released from one or more prostate protease nanosensors when exposed to an enzyme present in the prostate of the subject.

In some aspects, the disclosure provides a method comprising administering to a subject a prostate protease nanosensor or a composition as described by the disclosure, analyzing a biological sample from the subject, wherein the biological sample is not a derived from the prostate of the subject, and determining whether the detectable marker is present in the biological sample, wherein the presence of the detectable marker in the biological sample is indicative of the enzyme being present in an active form within the prostate of the subject.

In some embodiments, a subject is a mammalian subject, such as a human, dog, mouse, etc. In some embodiments, a subject has or is suspected of having cancer, such as prostate cancer. In some embodiments, an indolent cancer is non-metastatic cancer (e.g., indolent prostate cancer). In some embodiments, indolent (e.g., non-metastatic) prostate cancer has a Gleason score of 6 or below. In some embodiments, an aggressive cancer is metastatic cancer (e.g., metastatic prostate cancer). In some embodiments, aggressive prostate cancer (e.g., metastatic prostate cancer) has a Gleason score between 7 and 10. In some embodiments, a biological sample is not a derived from the prostate of the subject (e.g., is derived or obtained from a location or tissue other than the prostate of a subject). In some embodiments, a biological sample is a non-invasively obtained sample, such as a urine sample. In some embodiments, a biological sample is an invasively obtained sample, for example a blood sample, or tissue sample.

In some embodiments of methods described by the disclosure, detecting (e.g., detecting the presence of, or quantifying detectable markers) comprises a method selected from mass spectrometry (e.g., liquid chromatography-mass spectrometry, LC-MS/MS), PCR analysis, DNA microarray, fluorescence analysis, a capture assay (e.g., immunoassays, such as ELISA, etc.), optical imaging, magnetic resonance (MR) imaging, positron emission tomography (PET) imaging, intraoperative imaging, or any combination thereof.

In some embodiments, methods described by the disclosure further comprise the step of classifying a subject as having prostate cancer based upon the presence of detectable markers in a biological sample (e.g., based on the presence of detectable markers released from prostate protease nanosensors by cancer-associated protease activity into the biological sample). In some embodiments, a subject is diagnosed as having indolent prostate cancer or aggressive prostate cancer based upon the presence (or in some cases absence) of detectable markers in the biological sample. For example, in some embodiments a subject is classified as having indolent prostate cancer based upon the presence of a detectable marker released from a nanosensor comprising a substrate having the sequence set forth as GPLGVRGKC (SEQ ID NO: 1), PLGVRGK (SEQ ID NO: 32), SGSKII (SEQ ID NO: 35), or GSGSKIIGGGC (SEQ ID NO: 3). In some embodiments a subject is classified as having aggressive prostate cancer based upon the presence of a detectable marker released from a nanosensor comprising a substrate having the sequence set forth as GGGSGRSANAKGC (SEQ ID NO: 2), SGRSANAK (SEQ ID NO: 34), LGPKGQT (SEQ ID NO: 33), or GGLGPKGQTGGC (SEQ ID NO: 4).

In some aspects, methods described by the disclosure further comprise the step of diagnosing a subject as having prostate cancer based upon the presence of the detectable markers in the biological sample.

In some embodiments, methods described by the disclosure further comprise the step of administering a prostate protease nanosensor or a composition as described herein to the subject. In some embodiments, administration of compositions is performed by injection.

In some embodiments, a subject diagnosed as having prostate cancer based on the presence of detectable markers in a biological sample is administered a therapeutic agent (e.g., a therapeutic agent to treat prostate cancer), undergoes a therapeutic intervention (e.g., surgery to remove a prostate tumor), or a combination thereof.

### BRIEF DESCRIPTION OF DRAWINGS

FIGS. 1A-1B show schematic representations of prostate protease nanosensors for detection of prostate cancer. FIG. 1A is a schematic depicting injection of barcoded nanosensors into a subject. After proteolysis of the substrates, mass-encoded reporters filter into the urine and can be analyzed (e.g., by LC-MS/MS) to provide signatures of prostate cancer aggressiveness. FIG. 1B is a schematic depicting pipeline development for nanosensor libraries for prostate cancer staging, going from (I) human transcriptomic data through (II) substrate screening, (III) mouse model validation, and (IV) ex vivo sample analysis.

FIG. 2 shows selection and testing of candidate protease biomarkers for prostate cancer. On the left, a pipeline for selecting protease biomarkers for prostate cancer is depicted. Data indicating fold-change in RNA expression from The Cancer Genome Atlas (TCGA) for PCa (prostate cancer) vs normal and Aggressive vs Indolent prostate cancer are shown on the right. Aggressive PCa was defined as Gleason Score 7-10 and Indolent PCa was defined as Gleason 6. Histogram on the bottom right shows low levels of protease inhibitors in prostate cancer samples.

FIG. 3 shows a schematic depicting a screening approach of substrates with recombinant proteases. Pre- and post-cleavage substrate sequences are represented by SEQ ID NOs: 5 and 6, respectively.

FIG. 4 shows results of screening for PCa-associated protease substrates. The orthogonal set of substrates was selected for follow on screening.

FIG. 5 shows testing of prostate protease nanosensors having different scaffolds (e.g., multi-arm PEG, 10 nm iron oxide nanoparticle (IONP), and 20 nm IONP). Data indicate that multivalent PEG had significantly greater accumulation in the prostate compared to the two iron oxide nanoparticles.

FIG. 6 shows data from a screen den dying a subset of particles with desired substrate specificities.

FIGS. 7A-7F show testing of prostate cancer (PCa) protease nanosensors in mouse models of 22Rv1 and PC3 prostate cancer. FIG. 7A shows examples of PCa-associated protease substrate sequences; SEQ ID NOs: 7-11 are listed, top to bottom. FIG. 7B is a schematic depicting a timeline for administration of the nanosensors to mice. FIG. 7C shows urine signal data (e.g., detection of detectable markers in a urine sample) for 22Rv1 xenograft mice (100 mm³ in volume) compared to healthy mice. FIG. 7D shows urine signal data (e.g., detection of detectable markers in a urine sample) for PC3 xenograft mice (100 mm³ in volume) compared to healthy mice, FIG. 7E shows a comparison of urine signal data of each substrate in PC3 vs 22Rv1. FIG. 7F shows a ROC curve classifier indicating that a subset of sensors are able to classify PC3 from 22Rv1.

FIG. 8 shows analysis of transcriptomic data via SAMseq.

FIGS. 9A-9B show candidate protease biomarkers. FIG. 9A shows detection of prostate protease nanosensors for Gleason 7-10 samples versus Gleason Score 6 samples. FIG. 9B shows biochemical recurrence (TCGA) of cancer be gene expression measurement of MMP11 and KLK14.

FIG. 10 shows luminescence data of substrates for candidate proteases in the presence or Thrombin (top) and uPA(PLAU) (bottom).

FIGS. 11A-11B show evaluation of sensors in aggressive/metastatic cell xenograft models. FIG. 11A shows RNA expression profiling data for cancer-associated proteases in several cell lines (MDA PCa 2b, VCaP, LnCAP FGC, 22Rv1, DU145, PC3, and NCI-H660).

FIG. 11B shows matrigel invasion assay data indicating characterization of indolent nd aggressive cancers by prostate protease nanosensors.

FIGS. 12A-12B show selection of multiplexed sensors to classify metastatic tumors (Tmet) from non-metastatic tumors (Tnon-met). FIG. 12A shows data from a substrate cleavage assay for several prostate-specific substrates (SB14, PB2. PB13, B7; SEQ ID NOs: 7-10, top to bottom). FIG. 12B is a schematic depicting urinary reporter, substrate, and scaffold portions of the prostate protease nanosensors.

FIG. 13 shows representative data indicating that rr en :lassify Tmet from Tnon-met and outperform serum PS A measurements.

FIGS. 14A-14B show representative data for assay development to measure protease activity in mouse (FIG. 14A) and human (FIG. 14B) tissue samples.

FIG. 15 is a schematic showing representative data for a human sample cleavage analysis. Data for protease sensor substrates Q7, Q3, PQ11, Q10, PQ14, PQ13, and others are shown.

FIG. 16 is a schematic depicting discovery of candidate protease biomarkers.

FIG. 17 shows nanosensor formulation for enhanced prostate accumulation. Data indicate that addition of tumor-penetrating peptides (TPPs) to protease nanosensors increased the limit of detection to tumors <5 mm in size (e.g., diameter).

FIG. 18 shows representative data for cancer detection in 22Rv1 xenograft mice.

### DETAILED DESCRIPTION

Aspects of the disclosure relate to methods and compositions for detecting and monitoring protease activity within the prostate as an indicator of certain disease states (e.g., metastatic cancers, non-metastatic cancers, etc.). The disclosure relates, in some aspects, to the discovery that delivery of certain protease nanosensors (e.g., prostate protease nanosensors) to a subject, for example to the prostate of a subject, enables minimally invasive classification of the state of a tumor (e.g., aggressive, indolent, metastatic, non-metastatic, etc.) in the prostate of the subject. Without wishing to be bound by any particular theory, protease nanosensors described herein can detect enzymatic activity in vivo and noninvasively quantify physiological processes by harnessing the capacity of the nanosensors to circulate and sense the local microenvironment (e.g., environment of the prostate) while providing a read-out (e.g., detection of a detectable marker) at a site that is remote (e.g., a urine sample) from the target tissue (e.g., prostate).

For instance, as described in the Examples section herein, prostate-specific (e.g., prostate cancer-specific) protease activity can be assessed in order to classify a cancer in a subject as aggressive (e.g., metastatic) or indolent (e.g., non-metastatic) with higher specificity than currently available diagnostic modalities, such as serum prostate-specific antigen (PSA) assays. Without wishing to be bound by any particular theory, the combination of a scaffold that enhances accumulation of the nanosensors in prostate tissue, and prostate-specific (e.g., prostate cancer-specific) substrates that interact with prostate proteases in situ result in molecules configured to produce populations of detectable markers (e.g., a detectable marker signature) that are indicative of whether the subject has a prostate cancer, and if so, whether the cancer is indolent or aggressive.

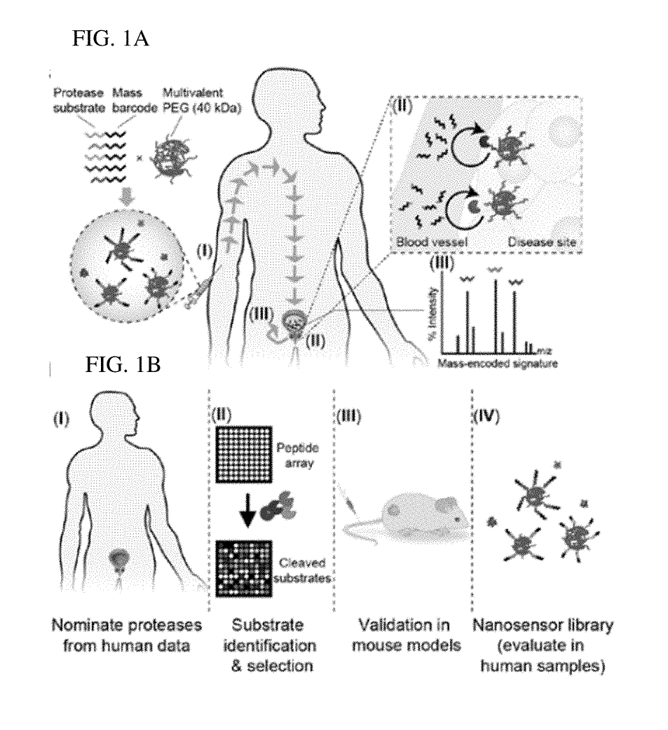

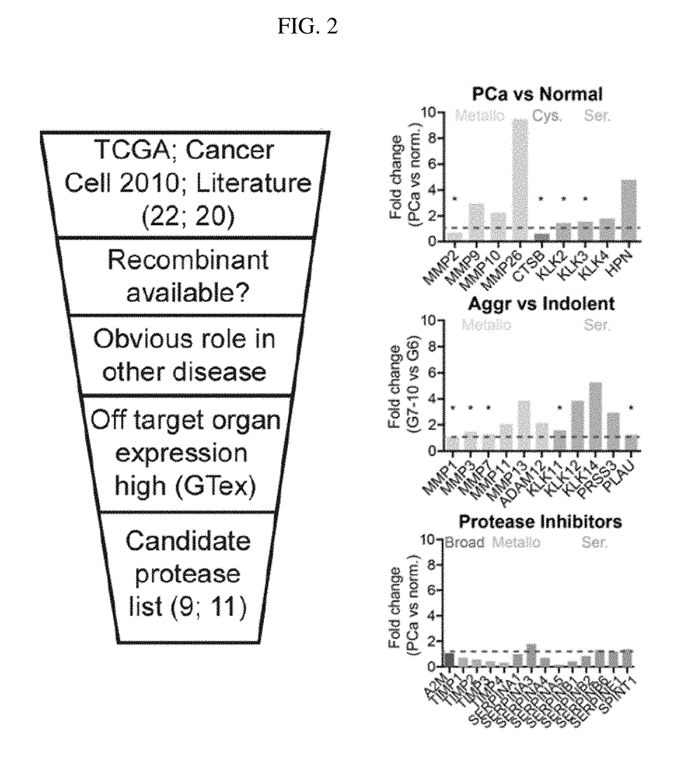

In some embodiments, the disclosure relates to the delivery of a set of protease-sensitive substrates (protease nanosensors) using scaffolds than enhance delivery of the nanosensors to the prostate of a subject. Upon encountering their cognate proteases, substrates are cleaved by endogenous enzymes (e.g., proteases) and reporter fragments are excreted into urine, providing a non-invasive diagnostic readout (FIG. 1). In some embodiments, the delivered nanosensors are responsive to proteases enriched in different stages of prostate tumor invasiveness (e.g., metastasis) and provide a high resolution, functionality driven snapshot of the prostate tumor microenvironment. Typically, aberrantly expressed proteases are candidate enzymes for cancer (e.g., prostate cancer) detection and analysis. Examples of prostate cancer-associated enzymes are described, for example, in FIG. 2.

Improved biomarkers are needed for prostate cancer, as the current gold standards have poor predictive value. Tests for circulating prostate-specific antigen (PSA) levels are susceptible to various noncancer comorbidities in the prostate and do not provide prognostic information, whereas physical biopsies are invasive, must be performed repeatedly, and only sample a fraction of the prostate. Injectable biosensors may provide a new paradigm for prostate cancer biomarkers by querying the status of the prostate via a noninvasive readout. Proteases are an important class of enzymes that play a role in every hallmark of cancer; their activities could be leveraged as biomarkers. A panel of prostate cancer proteases was identified through transcriptomic and proteomic analysis. Using this panel, a nanosensor library was developed that measures protease activity in vitro using fluorescence and in vivo using urinary readouts. In xenograft mouse models, this nanosensor library was applied to classify aggressive prostate cancer and to select predictive substrates. Last, a subset of nanosensors was coformulated with integrin-targeting ligands to increase sensitivity. These targeted nanosensors robustly classified prostate cancer aggressiveness and outperformed PSA. This activity-based nanosensor library could be useful throughout clinical management of prostate cancer, with both diagnostic and prognostic utility.

The lifetime risk for a US male to be diagnosed with prostate cancer is 1 in 6, yet mortality from this disease is only 1 in 35 (Prensner et al., Sci Transl Med 4:127rv3 (2012)). This discrepancy highlights the need for improved prognostication and management that could be enabled by accurate biomarkers (Sawyers, Nature 452:548-552 (2008)). While prostate-specific antigen (PSA) is the clinical blood biomarker standard, it is susceptible to various noncancer comorbidities. For example, infection and benign prostatic hyperplasia (BPH) are the most common sources of elevated PSA (Prensner et al., Sci Transl Med 4:127rv3 (2012)). Factors such as the time since a benign condition and PSA half-life impact the performance of this biomarker (Stamey et al., N Engl J Med 317:909-916 (1987), Nadler et al., J Urol 154:407-413 (1995)), which contributes to its poor predictive value: Only about 30% of men with elevated PSA have cancer detected upon biopsy (Prensner et al., Sci Transl Med 4:127rv3 (2012)). Further, biopsies sample only 1/1,000th of the prostate, which contributes to missing 30% of patients who bear high-grade cancer (Klotz et al., Nat Rev Clin Oncol 11:324-334 (2014)). Thus, a large fraction of the patients classified as low risk will progress and be at risk for recurrence. PCA3 is another biomarker that has been studied recently, but it is not as widely implemented and not recommended for use at the time of initial biopsy, according to National Comprehensive Cancer Network (NCCN) guidelines (Wei et al., J Clin Oncol 32:4066-4072 (2014)). Better biomarkers with lower susceptibility to benign false positives and improved ability to distinguish aggressive from indolent disease are needed. Aberrantly expressed proteases are candidates for cancer biomarkers, as they play critical roles in almost every hallmark of cancer (Dudani et al., Annu Rev Cancer Biol 2:353-376 (2018)). In fact, PSA is a protease in the Kallikrein family (KLK3), and is regulated by androgen signaling. KLK2, another member in the family, may also serve as a meaningful biomarker in prostate cancer, as demonstrated recently using a radiolabeled antibody to track androgen deprivation therapy (Thorek et al., Sci Transl Med 8:367ra167 (2016)). This strategy of imaging active proteases in prostate cancer has been applied to several other enzymes, such as urokinase plasminogen activator (uPA), which is up-regulated in aggressive prostate cancer (LeBeau et al., Cancer Res 75:1225-1235 (2015)). While these strategies show promise, they each only address one aspect of prostate cancer, such as imaging the androgen receptor axis. Additionally, the reliance on imaging as a read-out requires capital-intensive equipment and precludes simultaneous measurement of multiple enzymes. The ability to integrate multiple signals has shown significant promise in cancer diagnostics, such as the ConfirmMDx for Prostate Cancer (Partin et al., J Urol 192:1081-1087 (2014)), OncotypeDX Prostate Cancer assay (Eeden et al. Eur Urol 73:129-138 (2017)), and the Prolaris Prostate Cancer test (Cuzick et al., Lancet Oncol 12:245-255 (2011)), although these approaches require invasive biopsies. An ideal protease activity test would therefore integrate many prostate cancer-specific signals in a noninvasive platform.

As used herein, ABN refers to a protease nanosensor (e.g., a prostate protease nanosensor). This concept was applied to prostate cancer, with a focus on stratifying disease by first performing transcriptomic and proteomic analysis to identify prostate cancer-associated proteases overexpressed in cancer tissue relative to healthy tissue, as well as proteases that differentiate higher- and lower-grade cancers. Next, a panel of protease substrates was screened for activity against these disease-associated proteases and formulated a 19-plex ABN library. This library was evaluated using in vitro and in vivo models of human prostate cancer that recapitulated the protease expression patterns seen in human cancers. Finally, in some embodiments, nanosensors were modified with integrin-targeting peptides to enhance sensitivity and achieved robust classification of aggressive cancer and outperformed PSA for detection.

Accordingly, in some aspects, the disclosure provides a prostate protease nanosensor comprising a scaffold linked to a prostate-specific substrate, wherein the prostate-specific substrate includes a detectable marker, whereby the detectable marker is capable of being released from the prostate protease nanosensor when exposed to an enzyme present in a prostate (e.g., a prostate cancer-associated enzyme).

### Scaffolds

The prostate protease nanosensor comprises a modular structure having a scaffold linked to a protease-specific substrate (e.g., a prostate cancer-associated protease-specific substrate). A modular structure, as used herein, refers to a molecule having multiple domains.

The scaffold may include a single type of substrate, such as, a single type of protease-specific substrate (e.g., one or more substrates cleaved by the same protease), or it may include multiple types of different substrates (e.g., substrates cleaved by different proteases). For instance each scaffold may include a single (e.g., 1) type of substrate or it may include 2-1,000 different substrates, or any integer therebetween. Alternatively, each scaffold may include greater than 1,000 different substrates. Multiple copies of the prostate protease nanosensor are administered to the subject. In some embodiments, a composition comprising a plurality of different protease nanosensors (e.g. prostate protease nanosensors) may be administered to a subject to determine whether multiple enzymes and/or substrates are present. In that instance, the plurality of different protease nanosensors includes a plurality of detectable markers, such that each substrate is associated with a particular detectable marker or molecules.

The scaffold may serve as the core of the nanosensor. A purpose of the scaffold is to serve as a platform for the substrate and enhance delivery of the nanosensor to the prostate of the subject. As such, the scaffold can be any material or size as long as it can enhance delivery and/or accumulation of the nanosensors to the prostate of a subject. Preferably, the scaffold material is non-immunogenic, i.e. does not provoke an immune response in the body of the subject to which it will be administered. Non-limiting examples of scaffolds, include, for instance, compounds that cause active targeting to tissue, cells or molecules (e.g., targeting of nanosensors to the prostate), microparticles, nanoparticles, aptamers, peptides (RGD, iRGD, LyP-1, CREKA, etc.), proteins, nucleic acids, polysaccharides, polymers, antibodies or antibody fragments (e.g., herceptin, cetuximab, panitumumab, etc.) and small molecules (e.g., erlotinib, gefitinib, sorafenib, etc.). In some instances, a scaffold further comprises a tumor-penetrating peptide. In some instances, the tumor-penetrating peptide is iRGD, which may comprise CRGDKGPDC (SEQ ID NO: 36).

In some aspects, the disclosure relates to the discovery that delivery to the prostate of a subject is enhanced by protease nanosensors having certain polymer scaffolds (e.g., poly(ethylene glycol) (PEG) scaffolds). Polyethylene glycol (PEG), also known as poly(oxyethylene) glycol, is a condensation polymer of ethylene oxide and water having the general chemical formula HO(CH₂CH₂O)[n]H. Generally, a PEG polymer can range in size from about 2 subunits (e.g., ethylene oxide molecules) to about 50,000 subunits (e.g., ethylene oxide molecules. In some embodiments, a PEG polymer comprises between 2 and 10,000 subunits (e.g., ethylene oxide molecules).

A PEG polymer can be linear or multi-armed (e.g., dendrimeric, branched geometry, star geometry, etc.). In some embodiments, a scaffold comprises a linear PEG polymer. In some embodiments, a scaffold comprises a multi-arm PEG polymer. In some embodiments, a multi-arm PEG polymer comprises between 2 and 20 arms. Multi-arm and dendrimeric scaffolds are generally described, for example by Madaan et al. J Pharm Bioallied Sci. 2014 6(3): 139-150.

Additional polymers include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In some embodiments the polymers are polyesters, polyanhydrides, polystyrenes, polylactic acid, polyglycolic acid, and copolymers of lactic and glycoloic acid and blends thereof.

PVP is a non-ionogenic, hydrophilic polymer having a mean molecular weight ranging from approximately 10,000 to 700,000 and the chemical formula (C₆H₉NO)[n]. PVP is also known as poly[1-(2-oxo-1-pyrrolidinyl)ethylene], Povidone™, Polyvidone™, RP 143™, Kollidon™, Peregal ST™, Periston™, Plasdone™, Plasmosan™, Protagent™, Subtosan™, and Vinisil™. PVP is non-toxic, highly hygroscopic and readily dissolves in water or organic solvents.

Polyvinyl alcohol (PVA) is a polymer prepared from polyvinyl acetates by replacement of the acetate groups with hydroxyl groups and has the formula (CH₂CHOH)[n]. Most polyvinyl alcohols are soluble in water.

PEG, PVA and PVP are commercially available from chemical suppliers such as the Sigma Chemical Company (St. Louis, Mo.).

In certain embodiments the particles may comprise poly(lactic-co-glycolic acid) (PLGA). In some embodiments, a scaffold (e.g., a polymer scaffold, such as a PEG scaffold) has a molecular weight equal to or greater than 40 kDa. In some embodiments, a scaffold is a nanoparticle (e.g., an iron oxide nanoparticle, IONP) that is between 10 nm and 50 nm in diameter (e.g. having an average particle size between 10 nm and 50 nm, inclusive). In some embodiments, a scaffold is a high molecular weight protein, for example an Fc domain of an antibody.

As used herein the term “particle” includes nanoparticles as well as microparticles. Nanoparticles are defined as particles of less than 1.0 μm in diameter. A preparation of nanoparticles includes particles having an average particle size of less than 1.0 μm in diameter. Microparticles are particles of greater than 1.0 μm in diameter but less than 1 mm. A preparation of microparticles includes particles having an average particle size of greater than 1.0 μm in diameter. The microparticles may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter is about 5 nm to about 500 nm. In other embodiments, the diameter is about 100 nm to about 200 nm. In other embodiment, the diameter is about 10 nm to about 100 nm.

The particles may be composed of a variety of materials including iron, ceramic, metallic, natural polymer materials (including lipids, sugars, chitosan, hyaluronic acid, etc.), synthetic polymer materials (including poly-lactide-coglycolide, poly-glycerol sebacate, etc.), and non-polymer materials, or combinations thereof.

The particles may be composed in whole or in part of polymers or non-polymer materials. Non-polymer materials, for example, may be employed in the preparation of the particles. Exemplary materials include alumina, calcium carbonate, calcium sulfate, calcium phosphosilicate, sodium phosphate, calcium aluminate, calcium phosphate, hydroxyapatite, tricalcium phosphate, dicalcium phosphate, tricalcium phosphate, tetracalcium phosphate, amorphous calcium phosphate, octacalcium phosphate, and silicates. In certain embodiments the particles may comprise a calcium salt such as calcium carbonate, a zirconium salt such as zirconium dioxide, a zinc salt such as zinc oxide, a magnesium salt such as magnesium silicate, a silicon salt such as silicon dioxide or a titanium salt such as titanium oxide or titanium dioxide. A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference).

The scaffold may be composed of inorganic materials. Inorganic materials include, for instance, magnetic materials, conductive materials, and semiconductor materials. In some embodiments, the scaffold is composed of an organic material (e.g., a biological material that enhances delivery of the nanosensor to the prostate of a subject).

In some embodiments, the particles are porous. A porous particle can be a particle having one or more channels that extend from its outer surface into the core of the particle. In some embodiments, the channel may extend through the particle such that its ends are both located at the surface of the particle. These channels are typically formed during synthesis of the particle by inclusion followed by removal of a channel forming reagent in the particle. The size of the pores may depend upon the size of the particle. In certain embodiments, the pores have a diameter of less than 15 microns, less than 10 microns, less than 7.5 microns, less than 5 microns, less than 2.5 microns, less than 1 micron, less than 0.5 microns, or less than 0.1 microns. The degree of porosity in porous particles may range from greater than 0 to less than 100% of the particle volume. The degree of porosity may be less than 1%, less than 5%, less than 10%, less than 15%, less than 20%, less than 25%, less than 30%, less than 35%, less than 40%, less than 45%, or less than 50%. The degree of porosity can be determined in a number of ways. For example, the degree of porosity can be determined based on the synthesis protocol of the scaffolds (e.g., based on the volume of the aqueous solution or other channel-forming reagent) or by microscopic inspection of the scaffolds post-synthesis.

The plurality of particles may be homogeneous for one or more parameters or characteristics. A plurality that is homogeneous for a given parameter, in some instances, means that particles within the plurality deviate from each other no more than about +/−10%, preferably no more than about +/−5%, and most preferably no more than about +/−1% of a given quantitative measure of the parameter. As an example, the particles may be homogeneously porous. This means that the degree of porosity within the particles of the plurality differs by not more than +/−10% of the average porosity. In other instances, a plurality that is homogeneous means that all the particles in the plurality were treated or processed in the same manner, including for example exposure to the same agent regardless of whether every particle ultimately has all the same properties. In still other embodiments, a plurality that is homogeneous means that at least 80%, preferably at least 90%, and more preferably at least 95% of particles are identical for a given parameter.

The plurality of particles may be heterogeneous for one or more parameters or characteristics. A plurality that is heterogeneous for a given parameter, in some instances, means that particles within the plurality deviate from the average by more than about +/−10%, including more than about +/−20%. Heterogeneous particles may differ with respect to a number of parameters including their size or diameter, their shape, their composition, their surface charge, their degradation profile, whether and what type of agent is comprised by the particle, the location of such agent (e.g., on the surface or internally), the number of agents comprised by the particle, etc. The disclosure contemplates separate synthesis of various types of particles which are then combined in any one of a number of pre-determined ratios prior to contact with the sample. As an example, in one embodiment, the particles may be homogeneous with respect to shape (e.g., at least 95% are spherical in shape) but may be heterogeneous with respect to size, degradation profile and/or agent comprised therein.

Particle size, shape and release kinetics can also be controlled by adjusting the particle formation conditions. For example, particle formation conditions can be optimized to produce smaller or larger particles, or the overall incubation time or incubation temperature can be increased, resulting in particles which have prolonged release kinetics.

The particles may also be coated with one or more stabilizing substances, which may be particularly useful for long term depoting with parenteral administration or for oral delivery by allowing passage of the particles through the stomach or gut without dissolution. For example, particles intended for oral delivery may be stabilized with a coating of a substance such as mucin, a secretion containing mucopolysaccharides produced by the goblet cells of the intestine, the submaxillary glands, and other mucous glandular cells.

To enhance delivery the particles may be incorporated, for instance, into liposomes, virosomes, cationic lipids or other lipid based structures. The term “cationic lipid” refers to lipids which carry a net positive charge at physiological pH. Such lipids include, but are not limited to, DODAC, DOTMA, DDAB, DOTAP, DC-Chol and DMRIE. Additionally, a number of commercial preparations of cationic lipids are available. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA). A variety of methods are available for preparing liposomes e.g., U.S. Pat. Nos. 4,186,183, 4,217,344, 4,235,871, 4,261,975, 4,485,054, 4,501,728, 4,774,085, 4,837,028, 4,946,787; and PCT Publication No. WO 91/17424. The particles may also be composed in whole or in part of GRAS components. i.e., ingredients are those that are Generally Regarded As Safe (GRAS) by the US FDA. GRAS components useful as particle material include non-degradable food based particles such as cellulose. The scaffold can serve several functions. As discussed above, it may be useful for targeting the product to a specific region, such as a prostate (e.g., prostate tissue). In that instance, it could include a targeting agent such as a glycoprotein, an antibody, or a binding protein.

Further, the size of the scaffold may be adjusted based on the particular use of the protease nanosensor. For instance, the scaffold may be designed to have a size greater than 5 nm. Particles, for instance, of greater than 5 nm are not capable of entering the urine, but rather, are cleared through the reticuloendothelial system (RES; liver, spleen, and lymph nodes). By being excluded from the removal through the kidneys any uncleaved protease nanosensor will not be detected in the urine during the analysis step. Additionally, larger particles can be useful for maintaining the particle in the blood or in a tumor site where large particles are more easily shuttled through the vasculature. In some embodiments the scaffold is 500 microns-5 nm, 250 microns-5 nm, 100 microns-5 nm, 10 microns-5 nm, 1 micron-5 nm, 100 nm-5 nm, 100 nm-10 nm, 50 nm-10 nm or any integer size range therebetween. In other instances the scaffold is smaller than 5 nm in size. In such instance, the protease nanosensor will be cleared into the urine. However, the presence of free detectable marker (as opposed to uncleaved protease-specific substrate) can still be detected for instance using mass spectrometry. In some embodiments the scaffold is 1-5 nm, 2-5 nm, 3-5 nm, or 4-5 nm.

Optionally the scaffold may include a biological agent. In one embodiment, a biological agent could be incorporated in the scaffold or it may make up the scaffold. Thus, the compositions of the invention can achieve two purposes at the same time, the diagnostic methods and delivery of a therapeutic agent. In some embodiments the biological agent may be an enzyme inhibitor. In that instance the biological agent can inhibit proteolytic activity at a local site and the detectable marker can be used to test the activity of that particular therapeutic at the site of action.

### Substrates

The protease-specific substrate is a portion of the modular structure that is connected to the scaffold. A substrate (e.g., protease-specific substrate), as used herein, is the portion of the modular structure that promotes the enzymatic reaction in the subject (e.g., in the prostate of the subject), causing the release of a detectable marker. The substrate typically comprises an protease-sensitive portion (e.g., protease substrate) linked to a detectable marker.

The substrate is dependent on enzymes that are active in a specific disease state (e.g., prostate cancer, such as aggressive prostate cancer or indolent prostate cancer). For instance, tumors are associated with a specific set of enzymes. If the disease state being analyzed is a tumor, then a nanosensor is designed with one or more substrates that match those of the enzymes expressed by the tumor or other diseased tissue. Alternatively, the substrate may be associated with enzymes that are ordinarily present but are absent in a particular disease state. In this example, a disease state would be associated with a lack or signal associated with the enzyme, or reduced levels of signal compared to a normal reference.

An enzyme, as used herein refers to any of numerous proteins produced in living cells that accelerate or catalyze the metabolic processes of an organism. Enzymes act on substrates. The substrate binds to the enzyme at a location called the active site just before the reaction catalyzed by the enzyme takes place. Enzymes include but are not limited to proteases, glycosidases, lipases, heparinases, phosphatases.

In some embodiments, a substrate comprises an amino acid sequence that is cleaved by a protease (e.g., a protease-specific substrate). In some embodiments, the protease-specific substrate comprises an amino acid sequence cleaved by a serine protease, cysteine protease, threonine protease, aspartic protease, glutamic protease, or a metalloprotease. Examples of serine protease substrates include but are not limited to SLKRYGGG (SEQ ID NO: 12; plasma kallikrein) and AAFRSRGA (SEQ ID NO: 13; kallikrein 1). Examples of cysteine protease substrates include but are not limited to xxFRFFxx (SEQ ID NO: 14; cathepsin B), QSVGFA (SEQ ID NO: 15; cathepsin B), and LGLEGAD (SEQ ID NO: 16; cathepsin K). A non-limiting example of a threonine protease substrate is GPLD (SEQ ID NO: 17; subunit beta 1c). Examples of aspartic protease substrates include but are not limited to LGVLIV (SEQ ID NO: 18;

cathepsin D) and GLVLVA (SEQ ID NO: 19; cathepsin E. Examples of metalloprotease substrates include but are not limited to PAALVG (SEQ ID NO: 20; MMP2) and GPAGLAG (SEQ ID NO: 21; MMP9).

The substrate may be optimized to provide both high catalytic activity (or other enzymatic activity) for specified target enzymes but to also release optimized detectable markers for detection. Patient outcome depends on the phenotype of individual diseases at the molecular level, and this is often reflected in expression of enzymes. The recent explosion of bioinformatics has facilitated exploration of complex patterns of gene expression in human tissues (Fodor, S.A. Massively parallel genomics. Science 277, 393-395 (1997)). Sophisticated computer algorithms have been recently developed capable of molecular diagnosis of tumors using the immense data sets generated by expression profiling (Khan J, Wei J S, Ringner M, Saal L H, Ladanyi M, Westermann F, et al. Classification and diagnostic prediction of cancers using gene expression profiling and artificial neural networks. Nat Med 2001;7:673-679.). This information can be accessed in order to identify enzymes and substrates associated with specific diseases. Based on this information the skilled artisan can identify appropriate enzyme or substrates to incorporate into the biomarker nanoparticle.

In some embodiments, the substrate is cleaved by a protease associated with prostate cancer. In some embodiments, the protease associated with prostate cancer is associated with aggressive prostate cancer or metastatic prostate cancer, for example prostate cancer characterized by a Gleason score greater than 6 (e.g., a Gleason score between 7 and 10). In some embodiments, the protease associated with prostate cancer is associated with indolent prostate cancer or non-metastatic prostate cancer, for example prostate cancer characterized by a Gleason score of 6 or lower. Table 1 provides a non-limiting list of enzymes associated with (either increased or decreased with respect to normal) cancer. Numerous other enzyme/substrate combinations associated with specific diseases or conditions are known to the skilled artisan and are useful according to the invention.

In some embodiments, the substrate is a prostate cancer-specific substrate. As used herein, “prostate cancer-specific substrate” refers to an protease-specific substrate that is capable of being cleaved by a protease that is present (or upregulated) in the prostate of a subject having a disease (e.g., cancer). Examples of prostate cancer substrates include but are not limited to substrates targeted by MMP11, MMP13, KLK2, KLK3, KLK4, KLK5, KLK12, KLK14, PRSS3, uPA, MMP3, MMP26, and HPN. In some embodiments, prostate cancer substrates are targeted by MMP26, MMP10, HPN, MMP9, MMP11, KLK12, KLK14, KLK4, KLK3, KLK2, MMP13, KLK7, MMP3, ADAM12, PRSS3, and/or uPA. In some embodiments, aggressive or metastatic prostate cancer substrates are targeted by PRSS3, uPA, ADAM12, KLK7, MMP3, MMP13, KLK12, KLK14, and/or MMP11.

In some embodiments, certain cancers (e.g. aggressive or metastatic prostate cancers) are associated with upregulation of specific enzymes, for example KLK2, KLK5, KLK12, KLK14, MMP3, MMP11, MMP13, PRSS3, and/or uPA.

In certain embodiments, prostate cancer (e.g., prostate adenocarcinoma) is associated with upregulation of MMP26, MMP10, HPN, MMP9, MMP11, KLK12, KLK14, KLK4, KLK3, KLK2, MMP13, KLK7, MMP3, ADAM12, PRSS3, and/or uPA. In certain embodiments, prostate cancer (e.g., prostate adenocarcinoma) is associated with upregulation of HPN, KLK2, KLK3, KLK4, MMP9, MMP10, MMP26, KLK12, KLK14, PRSS3, uPA, and/or MMP11. In certain embodiments, MMP26, MMP10, HPN, MMP9, MMP11, KLK12, KLK14, KLK4, KLK3, KLK2, MMP13, KLK7, MMP3, ADAM12, PRSS3, and/or uPA is upregulated in a biological sample from a subject with prostate cancer compared to a biological sample from a subject without prostate cancer or compared to a non-cancerous biological sample. In certain embodiments, HPN, KLK2, KLK3, KLK4, MMP9, MMP10, MMP26, KLK12, KLK14, PRSS3, uPA, and/or MMP11 is upregulated in a biological sample from a subject with prostate cancer compared to a biological sample from a subject without prostate cancer or compared to a non-cancerous biological sample.

In certain embodiments, aggressive or metastatic prostate cancer is associated with upregulation of ADAM12, KLK12, KLK14, KLK7, MP11, MMP13, MMP3, PRSS3, and/or uPA. In certain embodiments, ADAM12, KLK12, KLK14, KLK7, MP11, MMP13, MMP3, PRSS3, and/or uPA is upregulated in a biological sample from a subject with aggressive or metastatic prostate cancer compared to a biological sample from a subject without aggressive or metastatic prostate cancer or compared to a non-metastatic biological sample. In some embodiments, aggressive or metastatic prostate cancers are associated with enzymes that cleave a substrate having the sequence set forth as GGGSGRSANAKGC (SEQ ID NO: 2), LGPKGQT (SEQ ID NO: 33), SGRSANAK (SEQ ID NO: 34), or GGLGPKGQTGGC (SEQ ID NO: 4). In some embodiments, certain cancers (e.g. indolent or non-metastatic prostate cancers) are associated with upregulation of specific enzymes that cleave a substrate having the sequence set forth as GPLGVRGKC (SEQ ID NO: 1), PLGVRGK (SEQ ID NO: 32), SGSKII (SEQ ID NO: 35), or GSGSKIIGGGC (SEQ ID NO: 3). In some embodiments, a prostate protease nanosensor comprises a metastatic cancer-specific substrate, a non-metastatic cancer-specific substrate, or a combination of metastatic and non-metastatic cancer-specific substrates.

In some embodiments, a substrate sequence may comprise a spacer sequence. In some embodiments, a spacer sequence comprises at least one (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100) glycine. A spacer sequence may be located at the N-terminus of a substrate sequence, at the C-terminus of a substrate sequence, or any combination thereof.

A substrate may be attached directly to the scaffold. For instance it may be coated directly on the surface of microparticles using known techniques, or chemically bonded to a polymeric scaffold, such as a PEG scaffold (e.g., via a peptide bond). Additionally, the substrate may be connected to the scaffold through the use of a linker. As used herein “linked” or “linkage” means two entities are bound to one another by any physicochemical means. Any linkage known to those of ordinary skill in the art, covalent or non-covalent, is embraced. Thus, in some embodiments the scaffold has a linker attached to an external surface, which can be used to link the substrate. Another molecule can also be attached to the linker. In some embodiments, two molecules are linked using a transpeptidase, for example Sortase A. In some embodiments, a linker comprises a cysteine.

The substrate is preferably a polymer made up of a plurality of chemical units. A “chemical unit” as used herein is a building block or monomer which may be linked directly or indirectly to other building blocks or monomers to form a polymer (e.g., a multi-arm PEG scaffold).

### Detectable Markers

The detectable marker is capable of being released from the prostate protease nanosensor when exposed to an enzyme in vivo. The detectable marker once released is free to travel to a remote site for detection. A remote site is used herein to refer to a site in the body that is distinct from the bodily tissue housing the enzyme where the enzymatic reaction occurs. In some embodiments, the bodily tissue housing the enzyme where the enzymatic reaction occurs is prostate tissue (e.g., prostate tumor tissue).

Modification of the protease-specific substrate by an enzyme in vivo, results in the production of a detectable marker (e.g., release or decoupling of the detectable marker from the scaffold upon cleavage of the protease-specific substrate by an enzyme). The detectable marker is a detectable molecule. It can be part of the substrate, e.g. the piece that is released or added upon cleavage or it can be a separate entity. In some embodiments, the detectable marker is composed of two ligands joined by a linker (e.g., a fluorescence resonance energy transfer (FRET) pair). The detectable marker may be comprised of, for instance one or more of a peptide, nucleic acid, small molecule, fluorophore/quencher, carbohydrate, particle, radiolabel, MRI-active compound, inorganic material, or organic material, with encoded characteristics to facilitate optimal detection, or any combination thereof. In some embodiments, the detectable marker comprises a GluFib peptide (SEQ ID NO: 22; EGVNDNEEGFFSAR) conjugated to a capture ligand and/or a fluorophore (e.g., a GluFib peptide flanked by a capture ligand, such as biotin, and a fluorophore, such as FAM).

In some embodiments, a prostate cancer-specific substrate comprises a capture ligand, which is a molecule that is capable of being captured by a binding partner. The detection ligand is a molecule that is capable of being detected by any of a variety of methods. While the capture ligand and the detection ligand will be distinct from one another in a particular detectable marker, the class of molecules that make us capture and detection ligands overlap significantly. For instance, many molecules are capable of being captured and detected. In some instances these molecules may be detected by being captured or capturing a probe. The capture and detection ligand each independently may be one or more of the following: a protein, a peptide, a polysaccharide, a nucleic acid, a fluorescent molecule, or a small molecule, for example. In some embodiments the detection ligand or the capture ligand may be, but is not limited to, one of the following: Alexa488, TAMRA, DNP, fluorescein, OREGON GREEN® (4-(2,7-difluoro-6-hydroxy-3-oxo-3H-xanthen-9-YL)isophthalic acid), TEXAS RED® (sulforhodamine 101 acid chloride), Dansyl, BODIPY® (boron-dipyrromethene), Alexa405, CASCADE BLUE® (Acetic acid, [(3,6,8-trisulfo-l-pyrenyl)oxy]-, 1-hydrazide, trisodium salt), Lucifer Yellow, Nitrotyrosine, HA-tag, FLAG-tag, His-tag, Myc-tag, V5-tag, S-tag, biotin or streptavidin.

In some embodiments, the capture ligand and a detection ligand are connected by a linker. The purpose of the linker is prevent steric hindrance between the two ligands. Thus, the linker may be any type of molecule that achieves this. The linker may be, for instance, a polymer such as PEG, a protein, a peptide, a polysaccharide, a nucleic acid, or a small molecule. In some embodiments the linker is a protein of 10-100 amino acids in length. In other embodiments the linker is GluFib (SEQ ID NO: 22; EGVNDNEEGFFSAR). Optionally, the linker may be 8 nm-100 nm, 6 nm-100 nm, 8 nm-80 nm, 10 nm-100 nm, 13 nm-100 nm, 15 nm-50 nm, or 10 nm-50 nm in length.

In some embodiments, the detectable marker is a ligand encoded reporter. Without wishing to be bound by any particular theory, a ligand encoded reporter binds to a target molecule (e.g., a target molecule present in a prostate), allowing for detection of the target molecule at a site remote from where the ligand encoded reporter bound to the target (e.g., at a sight remote from a prostate).

In some embodiments, a detectable marker is a mass encoded reporter, for example an iCORE as described in WO2012/125808, filed Mar. 3, 2012, the entire contents of which are incorporated herein by reference. Upon arrival in the diseased microenvironment, the iCORE agents interface with aberrantly active proteases to direct the cleavage and release of surface-conjugated, mass-encoded peptide substrates into host urine for detection by mass spectrometry (MS) as synthetic biomarkers of disease.

The detectable marker may be detected by any known detection methods to achieve the capture/detection step. A variety of methods may be used, depending on the nature of the detectable marker. Detectable markers may be directly detected, following capture, through optical density, radioactive emissions, non-radiative energy transfers, or detectable markers may be indirectly detected with antibody conjugates, affinity columns, streptavidin-biotin conjugates, PCR analysis, DNA microarray, optical imaging, magnetic resonance (MR) imaging, positron emission tomography (PET) imaging, intraoperative imaging, and fluorescence analysis.

A capture assay, in some embodiments, involves a detection step selected from the group consisting of an ELISA, including fluorescent, colorimetric, bioluminescent and chemiluminescent ELISAs, a paper test strip or lateral flow assay (LFA), bead-based fluorescent assay, and label-free detection, such as surface plasmon resonance (SPR). The capture assay may involve, for instance, binding of the capture ligand to an affinity agent.

The analysis (e.g., detecting) step may be performed directly on a biological sample (e.g., urine sample, blood sample, tissue sample, etc.) or the signature component may be purified to some degree first. For instance, a purification step may involve isolating the detectable marker from other components in a biological sample (e.g., urine sample, blood sample, tissue sample, etc.). Purification steps include methods such as affinity chromatography. As used herein an “isolated molecule” or “purified molecule” is a detectable marker that is isolated to some extent from its natural environment. The isolated or purified molecule need not be 100% pure or even substantially pure prior to analysis.

The methods for analyzing detectable markers by identifying the presence of a detectable marker may be used to provide a qualitative assessment of the molecule (e.g., whether the detectable marker is present or absent) or a quantitative assessment (e.g., the amount of detectable marker present to indicate a comparative activity level of the enzymes). The quantitative value may be calculated by any means, such as, by determining the percent relative amount of each fraction present in the sample. Methods for making these types of calculations are known in the art.

The detectable marker may be labeled. For example, a label may be added directly to a nucleic acid when the isolated detectable marker is subjected to PCR. For instance, a PCR reaction performed using labeled primers or labeled nucleotides will produce a labeled product. Labeled nucleotides (e.g., fluorescein-labeled CTP) are commercially available. Methods for attaching labels to nucleic acids are well known to those of ordinary skill in the art and, in addition to the PCR method, include, for example, nick translation and end-labeling.

Labels suitable for use in the methods of the present invention include any type of label detectable by standard means, including spectroscopic, photochemical, biochemical, electrical, optical, or chemical methods. Preferred types of labels include fluorescent labels such as fluorescein. A fluorescent label is a compound comprising at least one fluorophore. Commercially available fluorescent labels include, for example, fluorescein phosphoramidides such as fluoreprime (Pharmacia, Piscataway, N.J.), fluoredite (Millipore, Bedford, Mass.), FAM (ABI, Foster City, Calif.), rhodamine, polymethadine dye derivative, phosphores, Texas red, green fluorescent protein, CY3, and CY5. Polynucleotides can be labeled with one or more spectrally distinct fluorescent labels. “Spectrally distinct” fluorescent labels are labels which can be distinguished from one another based on one or more of their characteristic absorption spectra, emission spectra, fluorescent lifetimes, or the like. Spectrally distinct fluorescent labels have the advantage that they may be used in combination (e.g., “multiplexed”). Radionuclides such as 3H, 125I, 35S, 14C, or 32P are also useful labels according to the methods of the invention. A plurality of radioactively distinguishable radionuclides can be used. Such radionuclides can be distinguished, for example, based on the type of radiation (e.g. α, β, or δ radiation) emitted by the radionuclides. The 32P signal can be detected using a phosphoimager, which currently has a resolution of approximately 50 microns. Other known techniques, such as chemiluminescence or colormetric (enzymatic color reaction), can also be used.

Quencher compositions in which a “donor” fluorophore is joined to an “acceptor” chromophore by a short bridge that is the binding site for the enzyme may also be used. The signal of the donor fluorophore is quenched by the acceptor chromophore through a process believed to involve resonance energy transfer (RET). Cleavage of the peptide results in separation of the chromophore and fluorophore, removal of the quench, and generation of a subsequent signal measured from the donor fluorophore.

Methods The disclosure is based, in part, on delivery of certain protease nanosensors (e.g., prostate protease nanosensors) to a subject, for example to the prostate of a subject, for minimally invasive classification of the state of a tumor (e.g., aggressive, indolent, metastatic, non-metastatic, etc.) in the prostate of the subject. As used herein, “aggressive” prostate cancer refers to a prostate cancer having a Gleason score above 6, for example 7, 8, 9 or 10. In some embodiments, an aggressive prostate cancer is metastatic or is likely to become metastatic. An “indolent” prostate cancer refers to a prostate cancer having a Gleason score of 6 or below, for example, 6, 5, 4, 3, or 2. In some embodiments, an indolent prostate cancer is non-metastatic and is unlikely to become metastatic. It is useful to be able to differentiate non-metastatic primary tumors from metastatic tumors, because metastasis is a major cause of treatment failure in cancer patients. If metastasis can be detected early, it can be treated aggressively in order to slow the progression of the disease.

Accordingly, in some aspects, the disclosure provides a method for classifying cancer in a subject, the method comprising detecting in a biological sample obtained from a subject that has been administered a prostate protease nanosensor or composition as described herein (e.g., containing one or more prostate protease nanosensors), wherein the biological sample is not derived from the prostate of the subject, one or more detectable markers that have been released from one or more prostate protease nanosensors when exposed to an enzyme present in the prostate of the subject, and classifying the subject as having an indolent cancer or an aggressive cancer based on the identity of the detectable markers present in the biological sample, wherein the presence of the detectable markers in the biological sample is indicative of one or more cancer-associated enzymes being present in an active form within the prostate of the subject.

Compositions (e.g., prostate protease nanosensors) described herein can be administered to any suitable subject. As used herein, a subject is a human, non-human primate, cow, horse, pig, sheep, goat, dog, cat, or rodent. In all embodiments, male human subjects are preferred. In some embodiments, the subject preferably is a human suspected of having prostate cancer, or a human having been previously diagnosed as having prostate cancer.

As used herein, a biological sample is a tissue sample. The biological sample may be examined in the body, for instance, by detecting a label at the site of the tissue (e.g., by imaging the urine in the bladder of a subject). Alternatively the biological sample may be collected from the subject and examined in vitro (e.g., detecting a label at a site that is remote from the prostate of the subject). Biological samples include but are not limited to urine, blood, saliva, mucous secretion, and cell samples (e.g., buccal swabs, biopsy samples, etc.). In preferred embodiments the tissue sample is obtained non-invasively, such as by collecting the urine of the subject.

The prostate protease nanosensors of the disclosure are administered to the subject in an effective amount for detecting enzyme activity. An “effective amount”, for instance, is an amount necessary or sufficient to cause release of a detectable level of detectable marker in the presence of an enzyme. The effective amount of a composition described herein may vary depending upon the specific composition used, the mode of delivery of the composition, and whether it is used alone or in combination with other compounds (e.g., a composition comprising a multiplexed library of nanosensors or combined with administration of a therapeutic agent). The effective amount for any particular application can also vary depending on such factors as the disease being assessed or treated, the particular compound being administered, the size of the subject, or the severity of the disease or condition as well as the detection method. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective regimen can be planned.

Pharmaceutical compositions of the disclosure comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection.

Aspects of the disclosure relate to the discovery that, in some embodiments, prostate protease nanosensors circulate and sense the prostate microenvironment after systemic administration to a subject. In some embodiments, the systemic administration is injection, optionally subcutaneous injection. Preferably the material is injected into the body but could also be administered by other routes. For instance, the compounds of the present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

In some aspects, methods described by the disclosure comprise the step of diagnosing a subject has having prostate cancer based upon detection of detectable markers in a biological sample obtained from the subject after administration of the prostate protease nanosensors described herein. A “subject having or suspected of having prostate cancer” can be a subject that is known or determined to have cancerous prostatic cells or a prostatic tumor, or a subject exhibiting signs and symptoms of prostate cancer, including but not limited to burning or pain during urination, difficulty urinating, trouble stopping or starting while urinating, more frequent urges to urinate at night, loss of bladder control, decreased flow or velocity of urine stream, hematuria, etc. Subjects having or suspected of having cancer may be identified by various methods, including physical examination, subject's family medical history, subject's medical history, biopsy, ultrasonography, computed tomography, magnetic resonance imaging, magnetic resonance spectroscopy, positron emission tomography, or certain diagnostic tests (e.g., PSA assay).

In some embodiments, the disclosure relates to methods of treating prostate cancer in a subject comprising the step of administering a therapeutic agent (e.g., an agent for treatment of prostate cancer) to the subject or performing a therapeutic intervention on the subject who has been classified as having prostate cancer according the methods described herein. As used herein, “treat” or “treatment” refers to (a) preventing or delaying the progression of prostate cancer; (b) reducing the severity of prostate cancer; (c) reducing or preventing development of symptoms characteristic of prostate cancer; (d) preventing worsening of symptoms characteristic of prostate cancer; and/or (e) reducing or preventing recurrence of prostate cancer tumors or symptoms in subjects that were previously symptomatic for prostate cancer. Examples of therapeutic agents for the treatment of prostate cancer include but are not limited to abiraterone acetate, bicalutamide, cabazitaxel, degarelix, docetaxel, enzalutamide, flutamide, goserelin acetate, leuprolide acetate, mitoxantrone hydrochloride, nilutamide, sipuleucel-T, etc. Examples of therapeutic interventions for prostate cancer include surgery (e.g. surgery to remove a prostate tumor or prostate cancer cells, prostatectomy, etc.), radiation therapy, or a combination thereof.

### EXAMPLES

### Example 1

This example describes interrogation of protease activity in the prostate of patients after injection of biomarker nanoparticles and subsequent detection of reporter fragm.ents in remote samples (e.g., urine samples) (FIG. 1A). Proteases that are differentially unregulated in aggressive vs indolent cancer were identified, candidate substrates for the proteases were selected, and evaluated in mouse models of cancer and in human prostate samples (FIG. 1B).

Proteases that are upregulated in prostate cancer tissues versus normal adjacent, and in aggressive prostate cancer (e.g., high Gleason score, such as 7-10) vs indolent prostate cancer (e.g., low Gleason score, such as 6) were investigated and a set of proteases was selected (FIG. 2).

### Example 2

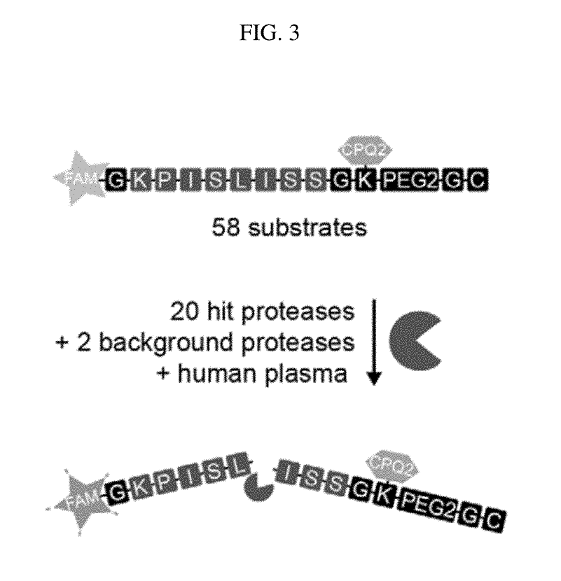

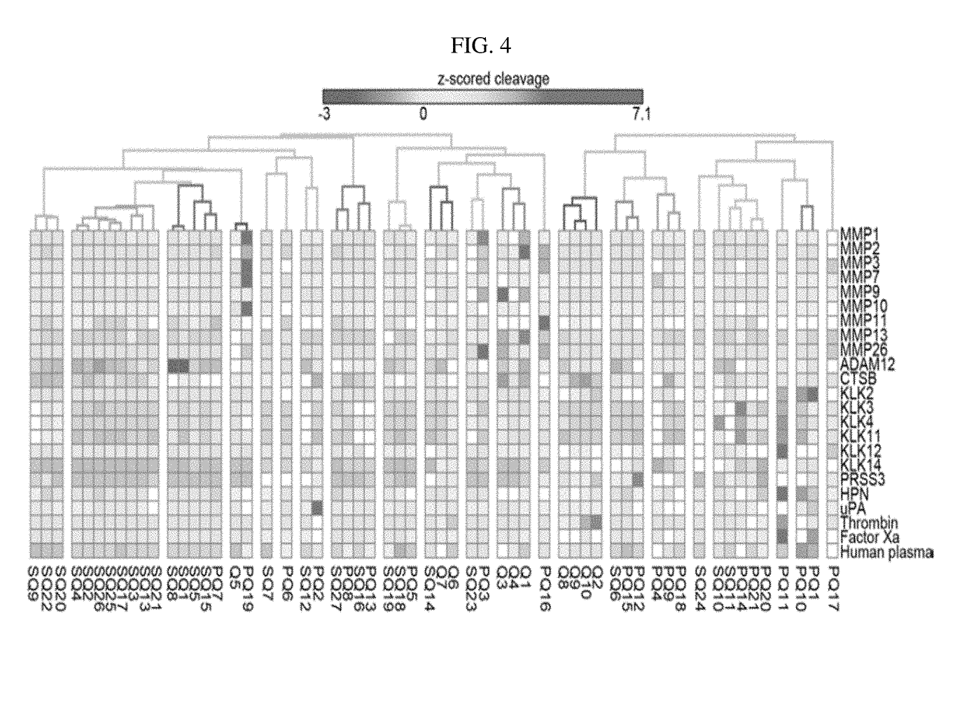

Using recombinant versions of the proteases selected from above, a panel of candidate substrates was screened to identify preferred protease substrates (FIGS. 3 and 4). These substrates were subsequently coupled to a nanocarier with the desirable pharmacokinetic properties (e.g., high prostate accumulation)

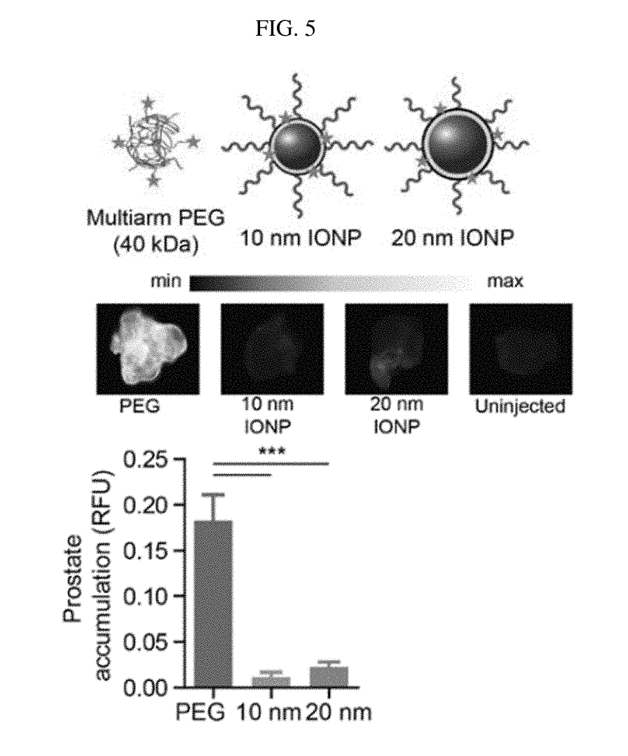

Prostatic accumulation of several potential scaffolds was tested. It was observed that 40 kDa PEG had significantly greater accumulation. in the prostate of healthy mice (FIG. 5). This scaffold was used for all in vivo experiments and follow on substrate screens.

The top 27 substrates identified from the first screen were coupled to scaffolds to identify a subset of twenty nanosensors for evaluation in a mouse model.

A subset of sensors was tested in mouse models of prostate cancer. One group of mice was bearing 22Rv1 xenografts and the other was bearing a xenograft of a more metastic cell line, PC3. With this subset, nanosensors were able to classify the more aggressive xenograft from the less aggressive xenograft.

### Example 3

This example describes classification of invasive prostate cancer using non-invasive methods through biomarker nanosensors. Proteolytic processes include protein degradation, post-translational modification, and signaling, which can lead to several hallmarks of cancer (Table 1).

TABLE 1
Examples of disease-associated (e.g., cancer-associated) proteases.

| Cancer Hallmark | Associated Proteases |
|---|---|
| Growth | MMP2, 3, 14 |
| | ADAM10, 17 |
| | KLK2, 3 |
| | CTSB, L, S |
| Survival & Death | MMP7, 9 |
| | ADAM10, 17 |
| | CTSB, S |
| Angiogenesis | MMP1, 2, 9 |
| | KLK1, 2, 6, 7 |
| | CTSB, S |
| Invasion & metastasis | MMP1, 14 |
| | CTSB, CTSL, CTSS |
| | KLK2, 3, 6 |
| | uPA HPN, ST14 |
| Inflammation | MMP8, ADAM17 |
| | CTSB, S |
| Immune evasion | MMP1, 3, 8, 9, 12 |
| | ADAM17 |
| | DPP4 |

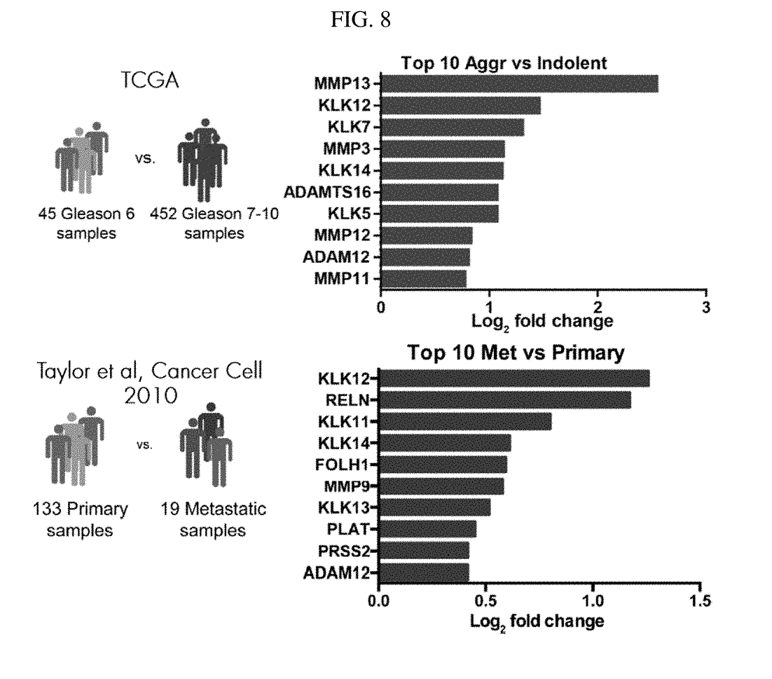

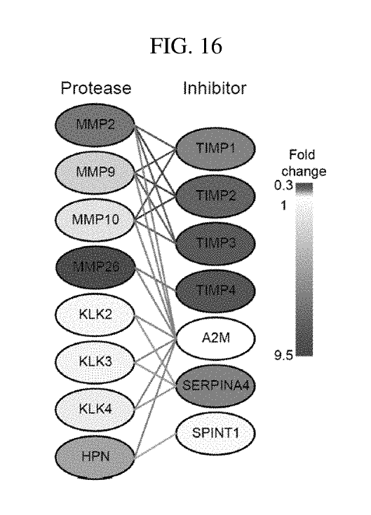

The general approach for identification of protease substrates for biomarker nanoparticles is to (I) ID proteases from human data, (II) formulate sensors (protease substrates and nanoparticle design), (III) evaluate sensors in mouse models, and (IV) perform translational development by analyzing human samples. To discover candidate protease biomarkers, transcriptomic data was analyzed via SAMseq (FIG. 8). Candidate proteases have various functions (for example as shown in Table 2 and FIG. 16).

TABLE 2
Protease functions.

| Protease | Function |
|---|---|
| MMP11 | Modulate cancer progression by remodeling |
| | ECM |
| MMP13 | Bone resorption (osteoclast) and bone |
| | deposition (osteoblast) |
| KLK12 | Carcinogenesis |
| KLK14 | Carcinogenesis |
| PRSS3 | Progression and metastasis in prostate cancer |
| uPA | Tissue differentiation and metastasis |

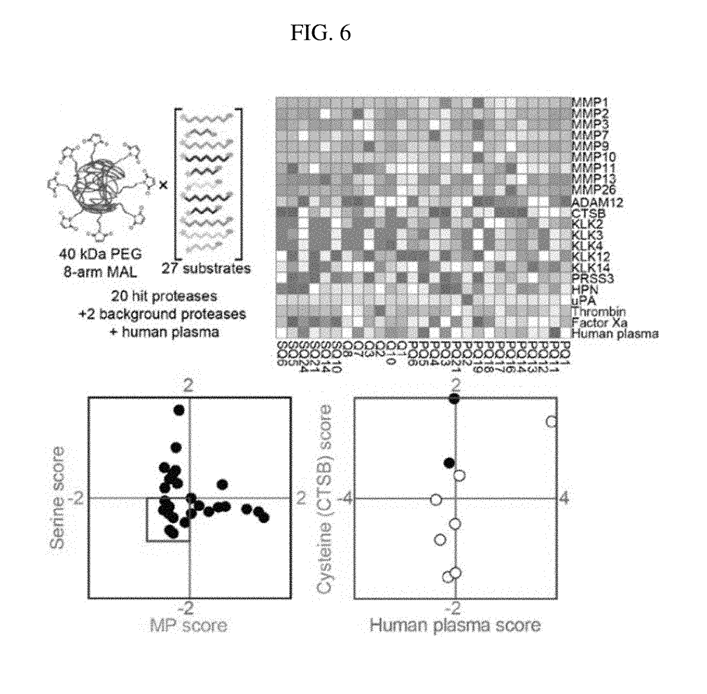

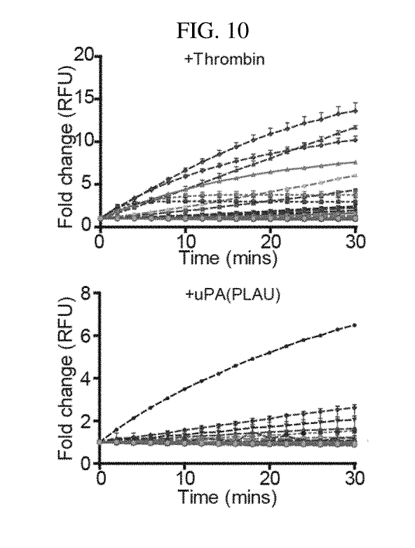

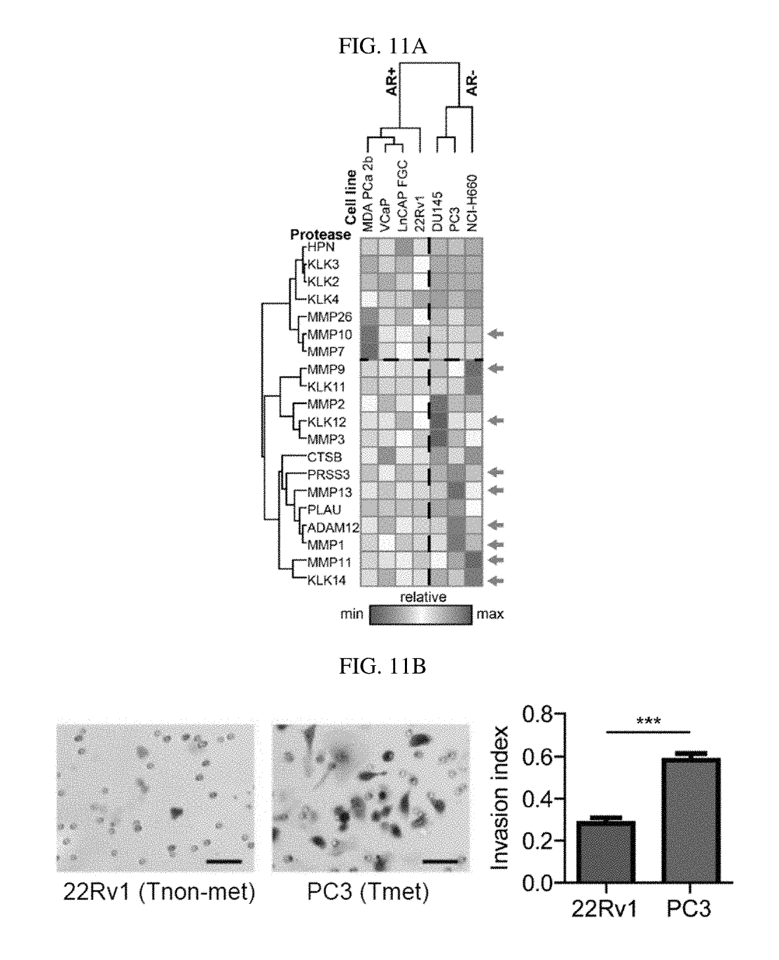

Substrates were developed for candidate proteases (FIG. 10). The most orthogonal set of substrates was selected (approximately 30), and then a second screen was performed to find a panel of 20 protease substrates that are responsive to the proteases of interest (FIG. 6). Nanosensors were formulated for enhanced prostate accumulation. The prostate is significantly smaller and less vascular than the spleen and liver, and it has dense fibromuscular stroma. It was observed that smaller nanoparticle formulations have improved prostate tumor accumulation and lower liver accumulation (FIG. 5). Sensors were evaluated in aggressive/metastatic cell line xenograft models. RNA expression was analyzed (FIG. 11A) and a matrigel invasion assay was performed to determine biomarker nanoparticle functionality (FIG. 11B).

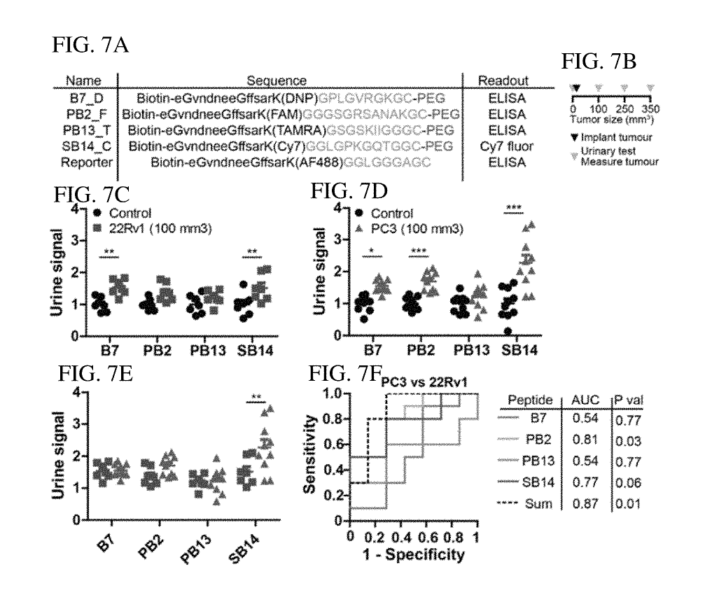

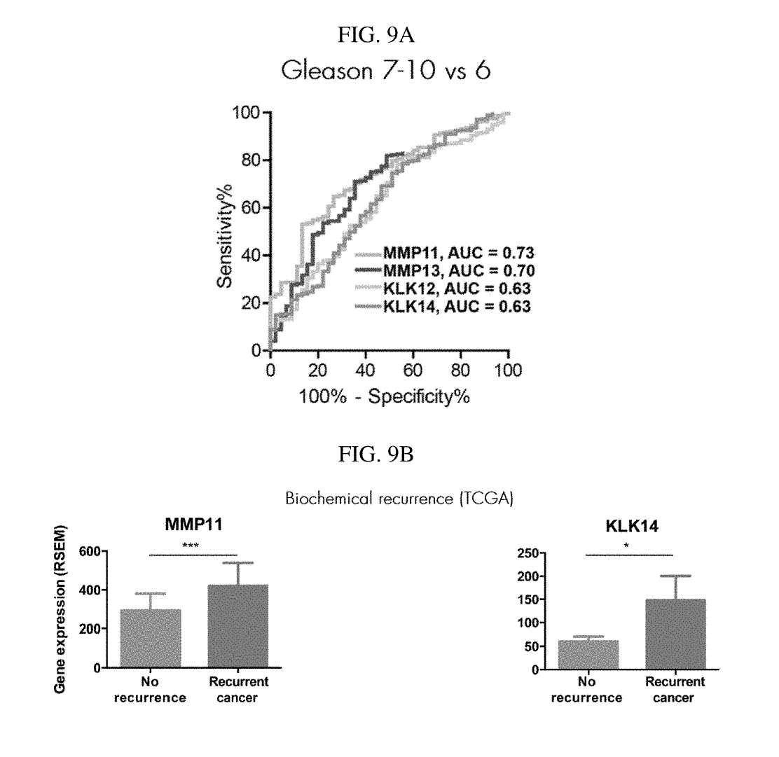

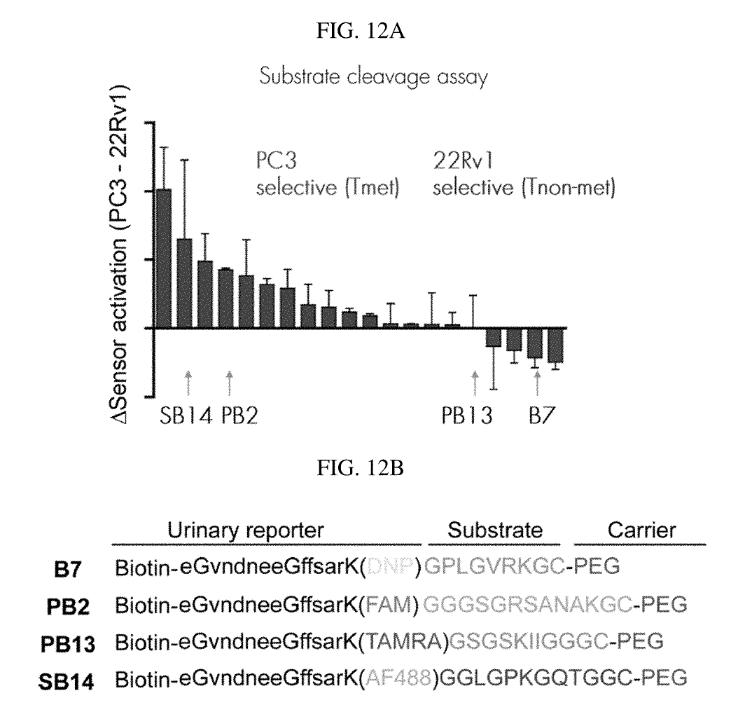

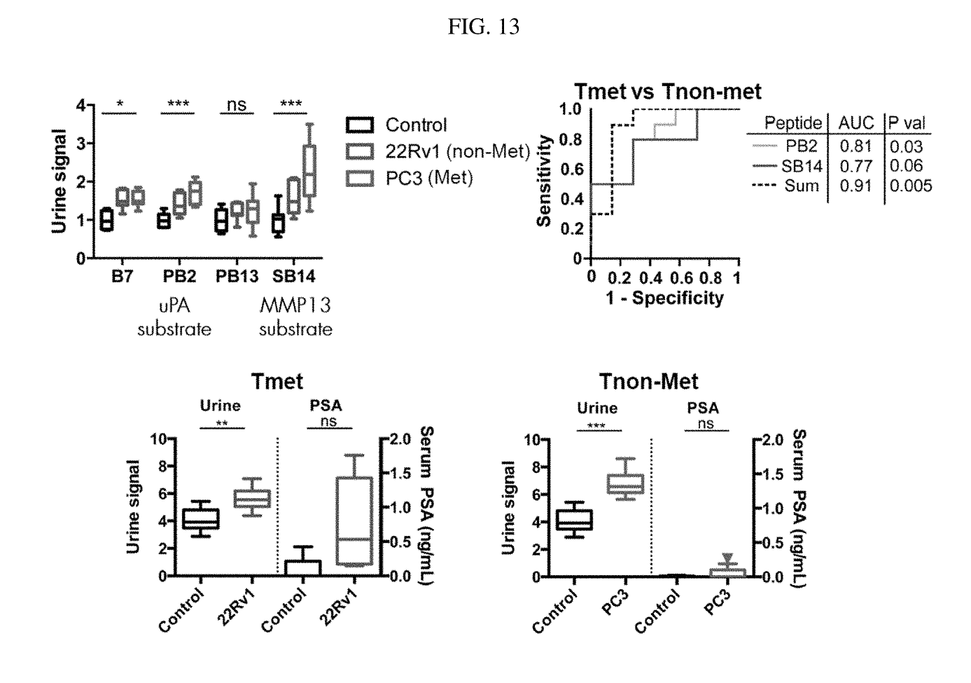

Xenograft model 22Rv1 (a non-metastatic tumor, referred to as Tₙₒₙ₋ₘₑₜ) is derived from a primary tumor. It is poorly differentiated, AR+, PSA+, and does not metastasize when implanted. PC3 (a metastatic tumor, referred to as Tₘₑₜ) is undifferentiated, AR⁻, PSA⁻, and metastasizes to lymph node (LN), lung, and bone. Multiplexed sensors were selected to classify Tₘₑₜ from Tₙₒₙ₋ₘₑₜ (FIGS. 7A-7F). A substrate cleavage assay was performed (FIG. 12A) with several protease nanosensors (FIGS. 9A-9B and FIGS. 12A-12B). Protease nanosensors were SB14 (MMP13 substrate, expressed by PC3; SEQ ID NO: 10), PB2 (uPA substrate, expressed by PC3; SEQ ID NO: 8), PB13 (broadly cleaved KLK substrate; SEQ ID NO: 9), and B7 (broadly cleaved MMP substrate; SEQ ID NO: 7). It was observed that multiplexed nanosensors classify Tₘₑₜ from Tₙₒₙ₋ₘₑₜ and outperform serum PSA measurements (FIG. 13) and that protease activity can classify Tₘₑₜ versus Tₙₒₙ₋ₘₑₜ cell lines. Multiplexing typically increases predictive power.

It was also observed that biomarker nanoparticles (e.g., detectable markers released from biomarker nanoparticles after protease cleavage) in cleared into the urine outperform standard measurement methods in both PSA-positive and PSA-negative tumors. 20-plex sensors were evaluated in GEMM to understand how proteolytic activity evolves through disease progression. Biomarkers of AR therapy response were evaluated in a LnCap xenograft model, for example as previously disclosed in Ellwood-Yen et al., Cancer Cell (2010).

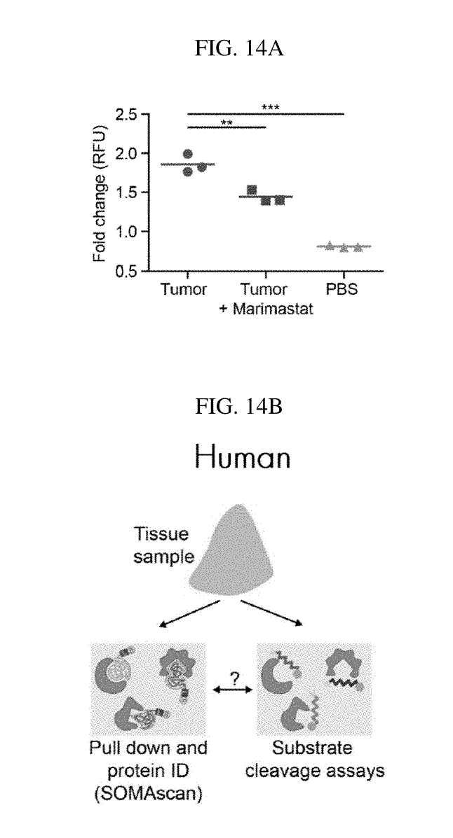

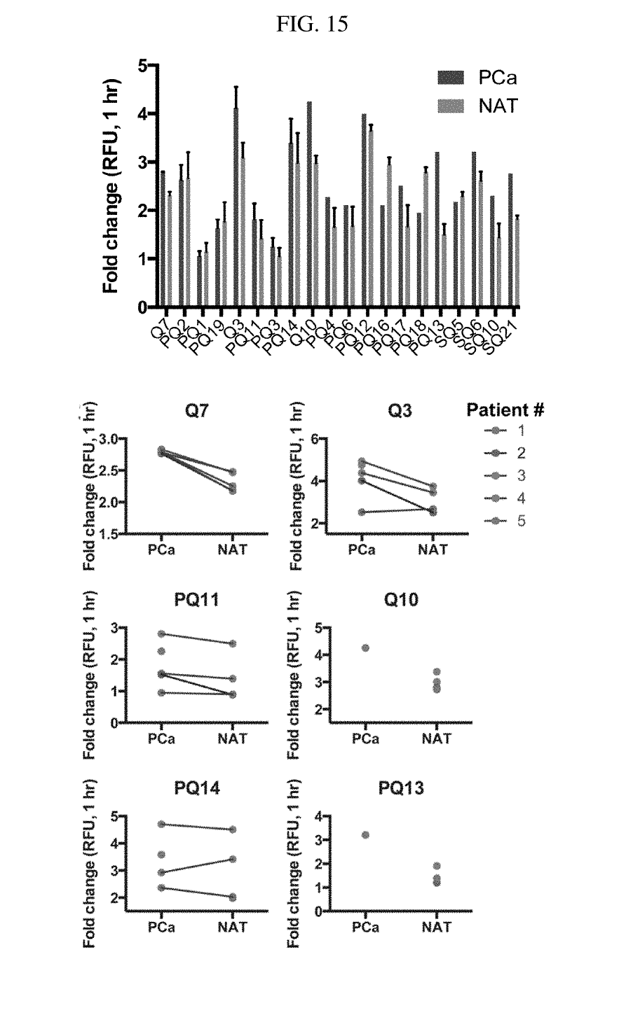

An assay was developed to measure protease activity in tissue samples. The mouse samples were excised, frozen in LN2, homogenized in PBS at 200 mgs/mL, and incubated with Q7 (FIG. 14A). In situ tissue zymography assays were additionally developed for frozen sections. Five sets of clinical human samples (both tumor and normal adjacent) were examined (FIG. 14B). The remaining fraction was tested on FRET substrates. Human sample cleavage was analyzed with substrate cleavage assays (Table 3). It was observed that many substrates are differentially cleaved in cancer tissue versus normal adjacent tissue. Table 4 and FIG. 15 shows discovery of candidate protease biomarkers.

TABLE 3
Human sample cleavage analysis.

| | | | | Percent | # of Substances |
|---|---|---|---|---|---|
| Patient | T | N | Gleason | Tumor | (Tumor, NAT) |
| 1 | T3a | Nx | 4 + 5 | 50 | 8, 20 |
| 2 | T2c | N0 | 3 + 3 | 30 | 6, 20 |
| 3 | T3a | N1 | 4 + 4 | 80 | 8, 13 |
| 4 | | | 4 + 3 | 70 | 8, 20 |
| 5 | T3b | N0 | 4 + 5 | 70 | 20, 4 |

TABLE 4
Protease functions.

| Protease | Function |
|---|---|
| MMP3 | Proliferation, metastasis, and apoptosis |
| MMP11 | Modulate cancer progression by remodeling |
| | ECM |
| MMP13 | Bone resorption (osteoclast) and bone |
| | deposition (osteoblast) |
| MMP26 | Involved in metastasis and apoptosis |
| KLK2 | Cleave PSA (KLK3) |
| KLK3 | Liquefy the seminal clot in the healthy prostate |
| KLK4 | Activator of uPA/PAR-1 signaling |
| KLK5 | Prostate cancer-associated serine protease |
| KLK12 | Carcinogenesis |
| KLK14 | Carcinogenesis |
| PRSS3 | Progression and metastasis in prostate cancer |
| uPA | Tissue differentiation and metastasis |
| HPN | Growth and progression of prostate cancer |

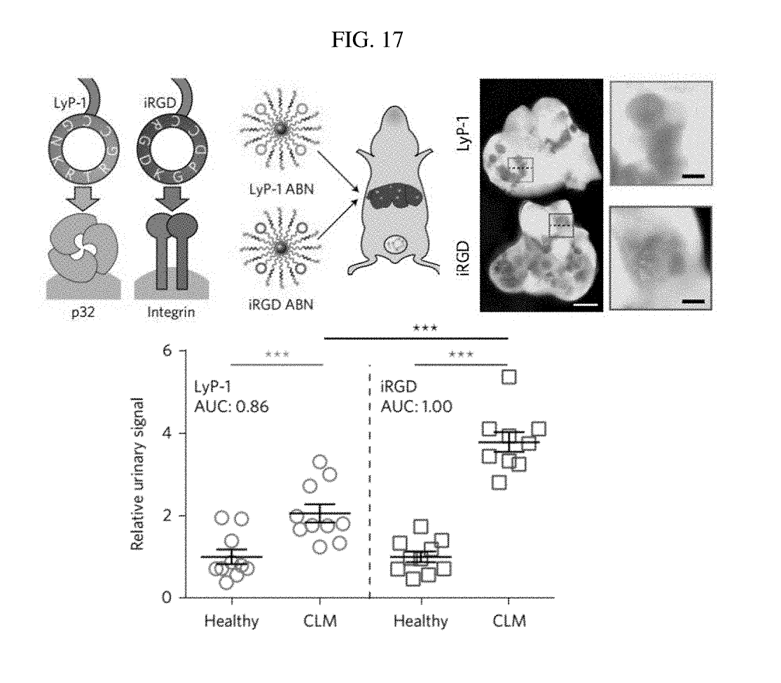

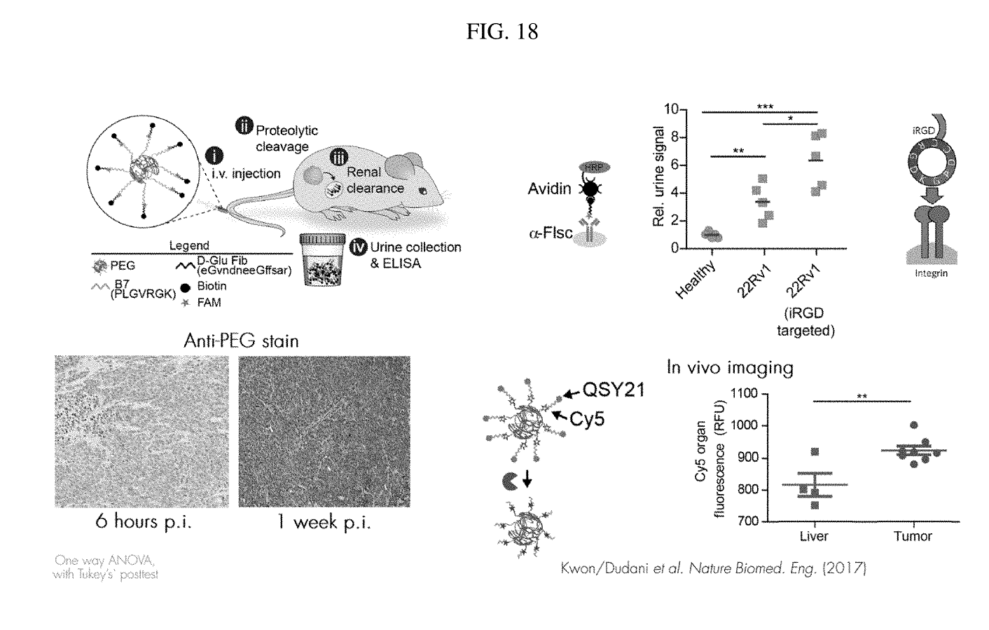

Substrates were developed for candidate proteases. Nanosensors were formulated for enhanced prostate accumulation. It was observed that the addition of tumor-penetrating peptides increased the limit of detection to <5 mm tumors (FIG. 17). FIG. 18 shows proof-of-concept for biomarker nanoparticle detection in 22Rv1 xenograft mice.

In the prostate inflammation model, mice develop prostatitis as they age, a common source of false positives. It has been observed that generally, biomarker signals either stayed the same or went down in older mice. Here, it was observed that sensors are not susceptible to this co-morbidity.

### Example 4

### Classification of Prostate Cancer Using a Protease Activity Nanosensor Library

Human transcriptome analysis identifies candidate protease biomarkers.

The goal was to systematically identify proteases expressed in human prostate cancer, formulate and build ABNs to measure their activity, and test the ABNs. The ABN platform comprises three components: a nanoparticle core that determines biodistribution and prevents urine accumulation of unliberated reporters, peptide substrates that are cleaved by target endoproteases, and urinary reporter barcodes paired to each substrate.

Transcriptomic data in The Cancer Genome Atlas (TCGA) was queried to identify proteases overexpressed in prostate cancer samples versus normal adjacent tissue (NAT) samples. Out of over 150 secreted and membrane-bound endoproteases in this dataset, 26 were expressed in tumors at levels at least 1.5-fold over NAT (panel termed “PRAD” to represent proteases overexpressed in prostate adenocarcinoma). Next, the same TCGA dataset was analyzed to identify proteases that differentiated Gleason 7 to 10 samples from lower-grade Gleason 6 samples because Gleason 6 lesions have been shown to lack many of the hallmarks of cancer (Klotz et al., Nat Rev Clin Oncol 11:324-334 (2014)). A list of 17 protease genes was elevated in the higher-scoring Gleason samples (panel termed “AGGR” to represent proteases overexpressed in aggressive cancer). Nine proteases were present on both lists. A subset of proteases from these analyses offered good classification potential, based on area under the receiver operating characteristic (AUROC) curve analysis, for distinguishing cancer from normal (max AUROC=0.93) and aggressive from indolent (max AUROC=0.73). These proteases were predominantly metalloproteinases (MPs) and serine proteases (SPs). Notably, the same TCGA samples were queried to look for concomitant protease inhibitor up-regulation and observed that many tissue inhibitors of MPs and serine protease inhibitors were expressed at reduced levels in cancer samples, highlighting broad proteolytic dysregulation. The protease lists were filtered based on several practical criteria, including availability of recombinant protease for use in substrate development, organ expression patterns using the Genotype-Tissue Expression (GTEx) portal, and knowledge of substrate specificities, resulting in a list of 14 candidate proteases.

Importantly, while patients with high expression of proteases identified from the cancer vs. normal (PRAD) analysis did not have poorer disease-free survival as quantified by

Kaplan-Meier analysis, patients with high expression of proteases in the AGGR list exhibited significantly poorer disease-free survival. This analysis underscores the importance of selecting biomarkers with good prognostic performance, rather than focusing solely on diagnosis. In an independent dataset (Taylor et al., Cancer Cell 18:11-22 (2010)), high expression of proteases in AGGR corroborated the same significantly poorer disease-free survival, further validating these biomarkers.

### Experimental Validation of Increased Protease Abundance and Activity in Human Prostate Cancer.

To confirm that the transcriptome-based candidates were expressed, a high throughput proteomics assay (SOMAscan) was applied (Mehan et al., PLoS One 7:e35157 (2012)). Five prostate tumor samples (Gleason sums from 6 to 9) and five matched NAT samples were analyzed for protein abundance, and the results were compared with the two sets of transcriptomic hits; any candidates that were not identified at the transcript level were also screened for. In the case of the PRAD list, all hits but one (KLK3, or PSA) were elevated in tumor samples compared with their average abundance in NAT, but no clear trends were observed in samples with higher Gleason scores. The lack of PSA protein elevation in the tumor samples highlights its poor performance as a biomarker to distinguish cancer from other conditions. In contrast, larger effect sizes were observed for the protein abundance of each of the proteases listed in the AGGR set, except for KLK7; these results mirrored the transcriptomic data, with clear differences in effect size observed in higher Gleason score tumors. Finally, the SOMAscan data identified two additional proteases (uPA and PRSS3) that were more abundant in the tumor samples. The modest effect sizes observed could be explained by the comparison with NAT samples, which includes reactive stroma, as well as the low tumor content in several samples. In this vein, a recent analysis of TCGA NAT samples relative to normal tissue (GTEx) demonstrated that NAT samples do not fully reflect normal tissue gene expression (Aran et al., Nat Commun 8:1077 (2017)).

To examine protein expression of candidate proteases in a tissue architecture-dependent method and compare abundance in inflamed tissue, one protease was selected from each list and type (MP and SP) and immunohistochemical (IHC) staining was performed on human prostate cancer tumor microarrays (TMAs). MMP26 and KLK14 stained positively in tumor samples, with a higher intensity of staining for KLK14. Notably, both proteases were expressed at elevated levels in tumors compared with normal, and with inflamed or hyperplastic samples; further, these proteases stained positive in sections from metastases.

Next, enzyme activity was sought to be analyzed in prostate cancer samples. Activity-based probes (ABPs) that specifically bind to active hydrolases are used to detect protease activity in human samples. Thus, a serine hydrolase probe, fluorophosphonate-TAMRA (FP-TAMRA; TAMRA is a fluorophore) was applied to fresh-frozen samples, which maintain proteolytic activity (Kwon et al., Nat Biomed Eng 1:0054 (2017); Withana et al., Nat Protoc 11:184-191 (2016)), and tumor cells were labeled in sections of a xenograft tumor derived from a human prostate cancer cell line, 22Rv1. This labeling was mitigated by the addition of a small molecule serine protease inhibitor called AEBSF. When applied to a fresh-frozen human prostate cancer TMA, FP-TAMRA labeled prostate cancer samples more than normal control samples.

As MP ABPs are less robust than serine ABPs, a FRET peptide substrate-cleavage assay was used to assay for MMP activity in the same tissue set evaluated by SOMAscan. Given the minimal tissue material available, each sample was evaluated with only a subset of substrates in duplicate. Multiple substrates were cleaved to a greater extent in tumor samples. Consistent with protein increases detected by SOMAscan, the cleavage signal elevation was modest, yet, in analyzing the 26 sets of paired measurements, significantly higher cleavage was detected in tumor samples. In the case of two MMP-sensing substrates, T7 and T3 (Kwong et al., Nat Biotechnol 31:63-70 (2013)), signal was elevated across the majority of tumor samples, indicating a pattern of increased MMP activity in prostate cancer.

Given the goal to establish an ABN library to both diagnose and classify prostate cancer, the analyses thus far were integrated and a list of 15 proteases was finalized upon which to build the nanosensor library (Table 5).

TABLE 5
Selected proteases for ABN development.

| Protease | Catalytic type | List | Transcrip | Protein |
|---|---|---|---|---|
| ADAM12 | Metallo | AGGR | Yes | Yes |
| HPN | Serine | PRAD | Yes | — |
| KLK2 | Serine | PRAD | Yes | — |
| KLK3 | Serine | PRAD | Yes | Yes |
| KLK4 | Serine | PRAD | Yes | Yes |
| KLK12 | Serine | Both | Yes | Yes |
| KLK14 | Serine | Both | Yes | Yes |
| MMP3 | Metallo | AGGR | Yes | Yes |
| MMP9 | Metallo | PRAD | Yes | Yes |
| MMP10 | Metallo | PRAD | Yes | Yes |
| MMP11 | Metallo | Both | Yes | — |
| MMP13 | Metallo | AGGR | Yes | Yes |
| MMP26 | Metallo | PRAD | Yes | Yes |
| PRSS3 | Serine | — | No | Yes |
| uPA | Serine | — | No | Yes |

### Development of Nanosensor Library Responsive to Selected Metalloproteinases and Serine Proteases.

With an identified set of MPs and SPs, a panel of substrates was developed to measure their activity. A panel of 58 FRET-paired peptide substrates (labeled T1-58-Q, where Q denotes quenched Table 6) was screened for cleavage by the 15 selected proteases. To account for background cleavage in circulation, Thrombin, Factor Xa, and human plasma were included as negative filters. The library comprised peptides with diverse physiochemical properties to provide broad coverage, and kinetic parameters of cleavage of the FRET-paired substrates by recombinant proteases were measured and z-score normalized by protease. Substrates were grouped by hierarchical clustering to remove substrates with overlapping cleavage patterns, as they would not provide any orthogonal insight, resulting in a down-selected panel of 26 substrates.

TABLE 6
Important peptides, nomenclature, and design.

| | | Readout |
|---|---|---|
| Name | Sequence | (sample |
| (T1-58)- | (5FAM)-(SUBSTRATE)-(CPQ2)- | Fluorescence |
| Q | (PEG2)-C | (in vitro/ |
| | | ex vivo) |
| -M | (Heavy isotope D-Glu-Fib)- | Urine |
| | (ANP)-(SUBSTRATE)-C | (LC-MS/MS) |
| T7-Q | (5FAM)-GGPLGVRGKK(CPQ2)- | Fluorescence |
| | (PEG2)-C (SEQ ID NO: 23) | (in vitro/ |
| | | ex vivo) |
| T7-QF | (QSY21)-GGPLGVRGKK(Cy5)- | Fluorescence |
| | (PEG2)-C (SEQ ID NO: 24) | (in vivo) |
| T7- | Biotin-eGvndneeGffsarK | ELISA |
| B(DNP) | (DNP)GPLGVRGKGC | (urine) |
| | (SEQ ID NO: 25) | |
| T7- | Biotin-eGvndneeGffsarK | ELISA |
| B(FAM) | (5FAM)GPLGVRGKGC | (urine) |
| | (SEQ ID NO: 26) | |
| T24-Q | (5FAM)-GGLGPKGQTGK(CPQ2)- | Fluorescence |
| | (PEG2)-C (SEQ ID NO: 27) | (in vitro/ |
| | | ex vivo) |
| T24- | Biotin-eGvndneeGffsarK | Fluorescence |
| B(Cy7) | (Cy7)GGLGPKGQTGGC | (urine) |
| | (SEQ ID NO: 28) | |
| T39-Q | (5FAM)-GGGSGRSANAKG- | Fluorescence |
| | K(CPQ2)-(PEG2)-GC | (in vitro/ |
| | (SEQ ID NO: 29) | ex vivo) |
| T39- | Biotin-eGvndneeGffsarK | ELISA |
| B(FAM) | (5FAM)GGGSGRSANAKGC | (urine) |
| | (SEQ ID NO: 30) | |
| ELISA | Biotin-eGvndneeGffsarK | ELISA |
| reporter | (AF488)GGLGGGAGC | (urine) |
| | (SEQ ID NO: 11) | |
| iRGD | C-PEG2-CRGDKGPDC | Tumor- |
| | (SEQ ID NO: 31); Cys2&3 | penetrating |
| | disulfide bridge | peptide |

Table 6 notes:
In most cases, peptide C terminus is CONH₂.
Lower case: D-stereoisomer.
Nomenclature: Q = quenched, B = biotin, M = mass-encoded.
For mass-encoding scheme.
FAM-CPQ2: FRET pair, with FAM as fluorophore and CPQ2 as quencher.
Cy5-QSY21: Red-shifted FRET pair.
Cy5: fluorophore pep,
QSY21: quencher;
order of fluorophore-quencher reversed in comparison to above.

5FAM, DNP, AF488 can be detected with an antibody; Cy7 measured by fluorescence.

For use in vivo, the peptides may be conjugated to a nanoparticle having robust accumulation in the prostate abilities. Thus, a biodistribution study was performed with three fluorophore-labeled carrier candidates and tested for their biodistribution following i.v. injection. Relative to two iron oxide carriers, a multivalent PEG polymer accumulated more in the prostate, and less in spleen and liver. Thus, the peptide substrates were conjugated to a PEG core and their cleavage profile was tested. While most substrates were cleaved similarly with and without PEG coupling, some discrepancies were observed, suggesting the need for empirical evaluation of peptide cleavage in any given formulation. Further mechanistic understanding of this variability may improve the development of ABN technology by identifying optimal surface presentation. Notably, analysis of the substrate cleavage profiles largely grouped MPs separately from SPs. Furthermore, MP and SP cleavage scores were calculated for each peptide and revealed an orthogonal pattern to their cleavage specificity: Peptides that were well cleaved by MPs were poorly cleaved by SPs. Some substrates were cleaved specifically by a single protease on the biomarker list, whereas others were cleaved by multiple or all members of the enzyme family tested (Table 7). Ultimately, all but two substrates that were poorly cleaved by both enzyme families were removed from the final panel to yield a 19-plex ABN library that offers broad coverage of relevant prostate cancer-expressed proteases, and thus should enable predictive signature building.

TABLE 7
Descriptive characteristics of ABN library.

| Name | Top hit | Second hit | Additional notes |
|---|---|---|---|
| PEG-T1-Q | MMP9 | — | HP |
| PEG-T2-Q | KLK2 | KLK3 | Thrombin |
| PEG-T3-Q | MMP9 | — | — |
| PEG-T7-Q | MMP9 | MMP26 | — |
| PEG-T20-Q | KLKs | — | — |
| PEG-T24-Q | MMP13 | — | — |
| PEG-T38-Q | KLK2 | — | FXa |
| PEG-T39-Q | uPA | — | — |
| PEG-T40-Q | MMP26 | ADAM12 | — |
| PEG-T41-Q | ADAM12 | — | Poorly cleaved |
| PEG-T43-Q | MMP3 | — | — |
| PEG-T48-Q | HPN | KLK2 | — |
| PEG-T49-Q | KLK14 | PRSS3 | — |
| PEG-T50-Q | KLK14 | KLK12 | — |
| PEG-T51-Q | KLK4 | KLK3 | — |
| PEG-T53-Q | MMP11 | MMP26 | Several MPs |
| PEG-T54-Q | ADAM12 | MMP13 | — |
| PEG-T56-Q | MMP10 | MMP3 | — |
| PEG-T58-Q | HPN | PRSS3 | Several SPs |

### Evaluation of ABN Library Against Cancer Cell Lines In Vitro and In Vivo.

The prostate cancer ABN library was first evaluated in vitro using human cell lines. To select representative models, protease gene expression was used across seven cancer cell lines from the Cancer Cell Line Encyclopedia (CCLE). Hierarchical clustering of these data grouped the cell lines based on androgen receptor status, showing that protease expression correlates with clinically meaningful prostate cancer status. It was noted that the PC3 cell line differentially expressed many of the proteases included in the AGGR list that discerns tumors by Gleason stage (Table 5). Further, the PC3 line is undifferentiated, AR−, PSA−, has metastatic potential, and is derived from a bone metastasis (Sobel et al., J Urol 173:342-359 (2005)). In contrast, the 22Rv1 cell line is poorly differentiated, AR+, PSA+, lacks metastatic potential, and is derived from serial passaging of a primary tumor. A transwell matrigel invasion assay was performed and it was observed that PC3 exhibits greater invasion capacity than 22Rv1, and was significantly inhibited by broad-spectrum protease inhibitors, suggesting this invasion was proteolytically driven.

Given their distinct protease profiles, the 22Rv1 and PC3 lines were selected to test the activity of the ABN library, and cleavage of the 19-plex fluorogenic ABNs was quantified in supernatant. Consistent with the library design, overall cleavage activity for both lines was reduced in the presence of marimastat (MMP inhibitor) or AEBSF (serine protease inhibitor), but not E64 (cysteine protease inhibitor). Additionally, there were cell line-specific cleavage patterns, with greater overall cleavage observed in the PC3 cells. To evaluate whether the panel of protease-responsive substrates can detect and classify disease in vivo, substrates were formulated with urinary reporters to generate in vivo ABNs. Based on previous work (Dudani et al., Adv Funct Mater 26:2919-2928 (2016); Warren et al., Proc Natl Acad Sci USA 111:3671-3676 (2014)), one ABN sensor was initially barcoded using a stable biotinylated D-stereoisomer of glutamate fibrinopeptide for detection (Table 6). These short peptides have been shown to reliably accumulate in the urine following proteolytic liberation from the carrier nanoparticle. The time point of urine collection was optimized by tracking urine signal generation in healthy mice and the optimal collection window was identified to be between 0 min and 60 min postinjection. Additionally, no difference was observed in signal when a second injection was administered to healthy mice 2 weeks later. In mice bearing tumor xenografts derived from 22Rv1 cells, the ABNs accumulated in the tumors. An increased urinary signal was detected from reporters liberated by proteolysis of the T7 substrate in 22Rv1 xenograft-bearing mice, and the performance of the sensor was equivalent when coupled to an alternately barcoded reporter. To confirm that the signal increase was due to proteolysis in the tumor, the protease activity of tumor homogenates was tested ex vivo and it was observed that T7 sensor cleavage was diminished in the presence of MMP inhibitor marimastat. An in vivo protease activity imaging study was also performed using a red-shifted FRET paired T7 substrate, which showed greater fluorescence signal in the tumor compared with the liver (Table 6).

Having achieved this proof-of-concept urine monitoring of protease activity with a single substrate, the entire ABN library was tested in vivo with an emphasis on identifying reporters to differentiate mice bearing more aggressive (PC3) versus less aggressive (22Rv1) xenografts. To quantify cleavage of the entire library in urine, the substrates were barcoded using a next generation of mass-encoded reporters built upon the isobar coded reporters method (Kwong et al., Nat Biotechnol 31:63-70 (2013)). This reengineered sensor library enables increased multiplexing by uniquely labeling each peptide with stable 13C and 15N atoms, allowing for quantitation of reporter barcodes across a large dynamic range using liquid chromatography-tandem mass spectrometry (Table 8).

TABLE 8
Multiplexed ABNs with mass-encoded barcodes.

| | Re- | Reporter | y6 | Peptide | PEG- | PEG- |
|---|---|---|---|---|---|---|
| Substrate | porter | parent | transition | MW | peptide | peptide |
| T40 | R3_01 | 789.3 | 683.8 | 2813 | 62504 | 90.4% |
| T2 | R3_02 | 789.3 | 685.8 | 2710.9 | 61687.2 | 94.0% |
| T3 | R3_03 | 789.3 | 687.8 | 2580.8 | 60646.4 | 95.6% |
| T7 | R3_04 | 789.3 | 689.8 | 2752 | 62016 | 92.4% |
| T20 | R3_05 | 789.3 | 691.8 | 2914.1 | 63312.8 | 94.0% |
| T24 | R3_06 | 792.3 | 689.8 | 2788.9 | 62311.2 | 94.9% |
| T38 | R3_08 | 792.3 | 693.8 | 3031.3 | 64250.4 | 43.6% |
| T39 | R3_09 | 792.3 | 695.8 | 2879 | 63032 | 93.7% |
| T41 | R3_10 | 792.3 | 697.8 | 2789 | 62312 | 93.3% |
| T1 | R3_11 | 795.3 | 695.8 | 2822.9 | 62583.2 | 95.8% |
| T43 | R3_12 | 795.3 | 697.8 | 3275.6 | 66204.8 | 94.1% |
| T48 | R3_13 | 795.3 | 699.8 | 2839 | 62712 | 94.3% |
| T49 | R3_14 | 795.3 | 701.8 | 2465.6 | 59724.8 | 90.2% |
| T50 | R3_15 | 795.3 | 703.8 | 2698.8 | 61590.4 | 90.7% |
| T51 | R3_16 | 798.3 | 701.8 | 2631.7 | 61053.6 | 92.0% |
| T53 | R3_17 | 798.3 | 703.8 | 2792.9 | 62343.2 | 96.0% |
| T54 | R3_18 | 798.3 | 705.8 | 3002 | 64017.6 | 62.1% |
| T56 | R3_19 | 798.3 | 707.8 | 2842.1 | 62736.8 | 92.0% |
| T58 | R3_20 | 798.3 | 709.8 | 2799.9 | 62399.2 | 96.2% |
| Reporter | R3_00 | 803.3 | 710.77 | 803.3 | — | — |

This new generation of reporters enables increased multiplexing with improved readouts by using five barcodes per parent mass, with parent masses spaced by 3 Da and y6 ions spaced by 2 Da. PEG-peptide conjugations were analyzed by reverse-phase HPLC and determined to be greater than 90% for most particles. A few had lower calculated purity due to polydisperse populations but still were conjugated to PEG.

To account for variability in glomerular filtration rate, urine volume, and hydration state, a free reporter (not coupled to PEG) was co-injected. The 19-plex ABN library was serially injected i.v. to PC3 tumor-bearing mice over the course of tumor development. As tumors increased in size, an increase in the aggregate urine signal was observed, expressed as the sum of all disease-sensitive reporters normalized to the co-administered free reporter.

Next, the ability of the 19-plex library to classify PC3 from 22Rv1 tumor-bearing mice was sought to be determined. Using the mass-encoded reporters to examine the cleavage of each individual sensor, an early time point was focused on and it was observed that several substrates were differentially cleaved between animals bearing similarly sized xenografts (˜100 mm3) from the more (PC3) versus less aggressive (22Rv1) cell lines. Overall, the cleavage profile differences between the two cohorts agree with each cell line's protease expression patterns and the substrate specificity of each protease. For example, substrates T24 and T39 show higher relative urine signal change in mice bearing PC3 xenografts compared with 22Rv1 xenografts; in vitro, these substrates are cleaved by proteases overexpressed in PC3 cells, MMP13 and uPA. Other substrate sensors that are predominantly cleaved by proteases expressed by 22Rv1 cells show preferential signal generation in 22Rv1-bearing mice; for example, T40 and T51 are cleaved by MMP26 and KLK4, respectively.

### An Integrin-Targeted ABN Library Subset Robustly Classifies Aggressive Prostate Cancer.

One advantage of a highly multiplexed library is the capacity to nominate a smaller subset of sensors for a specific application. The results of testing the 19-plex library in vitro (fluorogenic) and in vivo (mass-encoded) was integrated against PC3 and 22Rv1 cells to select a minimal subset of ABNs for a more practical diagnostic platform with simpler urinary readouts (Warren et al., Proc Natl Acad Sci USA 111:3671-3676 (2014)).

As stated above, urinary reporters released from T24 and T39 sensors, which are selectively cleaved by MMP13 and uPA, were elevated in PC3-bearing mice compared with 22Rv1 mice and were also cleaved differentially by PC3 cell supernatants in vitro. Consistent with this result, PC3 flank xenografts expressed MMP13 and uPA more than 22Rv1 flank xenografts. Interestingly, both of these proteases play a role in bone metastasis, which is the source of the PC3 cell line (Gartrell et al., Nat Rev Clin Oncol 11:335-345 (2014)), and also a common site of metastasis for prostate cancer. T7 was also nominated for the targeted ABN panel, as it gave rise to urine signals in both 22Rv1 and PC3 mice and was used in the earlier optimization experiments.

Noting that the effect sizes observed were small, consistent with the untargeted nature of the nanosensors, the performance of the selected subset of sensors was sought to be increased by using tumor-targeting peptides. It has previously been shown that adding integrin-targeting, tumor-penetrating peptides can increase performance of ABNs (Kwon et al., Nat Biomed Eng 1:0054 (2017)). A cyclic form of RGD, iRGD, enables greater tumor penetration and delivery by binding αvβ3/β5 integrins (Sugahara et al., Science 328:1031-1035 (2010)). After confirming that av integrins were overexpressed in human prostate cancer (Sutherland et al., Cancers (Basel) 4:1106-1145 (2012)) by staining a TMA, and that both PC3 and 22Rv1 xenografts stained for high levels of av integrins, the ABN design was modified to incorporate iRGD. It was initially tested whether coupling iRGD to the ABN increased performance of T7 nanosensors in mice bearing 22Rv1-derived xenografts at 100-mm³ aggregate tumor burden. The signal derived from iRGD-modified T7 ABNs was significantly greater than that produced by unmodified ABNs.

Guided by this positive test, a three-plex of iRGD-modified ABNs (iRGD-ABNs) was next produced using substrates T7, T24, and T39 (Table 6). To simplify urinalysis, these ABNs were designed to release biotinylated urinary reporters to enable ELISA-based readouts. Following i.v. injection of the three-plex iRGD-ABNs, the combined urine reporter signal was elevated in both 22Rv1-bearing and PC3-bearing animals compared with controls. Notably, this urine diagnostic sensor increase was both significant and more robust than serum PSA elevation in both cohorts. The pattern is more striking in PC3-bearing mice as this tumor is PSA negative, suggesting the combination of signal amplification from protease activity and concentration into urine concentration could be more predictive than serum biomarkers (Kwon et al., Nat Biomed Eng 1:0054 (2017)). Additionally, PSA measurements in mice may overestimate its sensitivity, as there is no mouse homolog of PSA (Lundwall et al., Biol Chem 387:243-249 (2006)).

It was next tested whether the three-plex iRGD-ABNs could classify distinct prostate cancer tumors. When the individual reporter readouts were compared, mice bearing PC3 tumors gave rise to significantly greater cleavage of both the uPA (T39) and MMP13 (T24) substrates relative to 22Rv1, consistent with the relative protease expression profile of the cell lines. Both sets of tumor-bearing mice generated T7 urine signals that were elevated relative to control animals, but this sensor readout did not classify between the two cohorts. Based on ROC curve analysis, the T39 and T24 ABNs classified the mice bearing the more aggressive PC3-derived tumors as distinct from 22Rv1-bearing mice. Importantly, the sum of the uPA and MMP13 substrate signals significantly increased the classification power of the nanosensors.

Finally, a common complication of existing prostate cancer biomarkers is the high rate of false positives due to comorbidities, such as BPH and prostatitis (Prensner et al., Sci Transl Med 4:127rv3 (2012)). It was sought to assess whether the three-plex ABNs were similarly susceptible to comorbidities by evaluating them in nonobese diabetic mice that develop prostatitis and also display prostatic hypertrophy as they age (Penna et al., J Immunol 179: 1559-1567 (2007); Jiang et al., Differentiation 82:220-236 (2011)). At 20 weeks of age, prostatic hyperplasia and immune cell infiltration were noted in the prostate, but urine signal was not elevated in the older mice, highlighting that these diagnostic tools are both sensitive and specific. This model represents an initial step toward defining the specificity of ABNs in animal models. This approach needs to be systemically evaluated in humans, but several reports are encouraging, such as evidence of increased uPA activity in cancer tissue versus BPH (Böhm et al., J Cancer Res Clin Oncol 139:1221-1228 (2013)) and elevated plasma levels of MMP13 and MMP9 in patients with cancer versus BPH (Morgia et al., Urol Res 33:44-50 (2005)).

### Discussion

A bottom-up approach was applied to design, build, and test a panel of ABNs to detect and classify prostate cancer. First, transcriptomic and proteomic tools were used to nominate proteases that identify and stratify prostate cancer in human samples. Next, substrates were designed to detect these proteases and an ABN library was built using these substrates. The resulting 19-plex ABN library was evaluated in vitro and in vivo using mass-encoded barcodes for urinary analysis in cell line xenograft models. A pair of proteases were identified that were differentially expressed in the PC3 cell line. To increase performance, a panel of ABNs was modified with iRGD to bind overexpressed integrins in prostate cancer. The iRGD-modified ABNs robustly classified invasive (PC3) from less invasive (22Rv1) tumor-bearing mice, and out-performed PSA as a diagnostic biomarker in these models. These ABNs did not produce false-positive results in a prostatitis mouse model.

Further reduction to the risk of false positive signals could be achieved by more in-depth benchmarking of net protease activity against BPH and other comorbidities. For example, a systematic evaluation of the degradome (proteases and inhibitors) of a range of tissue sources and contexts using a systems biology approach could be informative, and build upon the existing analysis to improve the specificity of the selected proteases used for prostate cancer detection.

### Methods

Transcriptomic, SOMAscan, and Activity Analysis.

Differential expression analysis was performed on TCGA data using SAMseq. Survival analysis was performed using cBioPortal. SOMAscan was performed at the Beth Israel Deaconess Medical Center (BIDMC) Genomics Proteomics Core. Fresh frozen prostate cancer tissue microarray was obtained from US BioChain (T6235201) and stained with FP-TAMRA (88318; Sigma) at 1 μM in PBS.
Animal Models. All animal studies were approved by Massachusetts Institute of Technology's Committee on Animal Care (CAC) (Protocol 0417-025-20). Four- to six-week-old male NCr nude mice (Taconic) were injected bilaterally with 3.5×106 PCa cells per flank in a 1:1 ratio of complete media and Matrigel (354234; Corning). Baseline urine measurement was obtained before xenograft implantation. Histology sectioning and staining was performed at KI Histology Core.

1. A prostate protease nanosensor comprising a scaffold linked to a prostate-specific substrate, wherein the prostate-specific substrate includes a detectable marker, whereby the detectable marker is capable of being released from the prostate protease nanosensor when exposed to an enzyme present in a prostate.

2. The nanosensor of claim 1, wherein the scaffold comprises a high molecular weight protein, a high molecular weight polymer, or a nanoparticle, optionally wherein the protein, polymer or nanoparticle is greater than about 40 kDa.

3-5. (canceled)

6. The nanosensor of claim 1, wherein each prostate-specific substrate comprises a cancer substrate, optionally wherein the cancer substrate is cleaved by an enzyme associated with prostate cancer.

7. The nanosensor of claim 6, wherein the cancer substrate is a substrate cleaved by an enzyme selected from MMP11, MMP13, KLK2, KLK3, KLK4, KLK5, KLK12, KLK14, PRSS3, uPA, MMP3, MMP26, HPN, MMP10, MMP9, ADAM12, or any combination thereof.

8. The nanosensor of claim 7, wherein the cancer substrate comprises the amino acid sequence set forth as GPLGVRGKC (SEQ ID NO: 1), GGGSGRSANAKGC (SEQ ID NO: 2), GSGSKIIGGGC (SEQ ID NO: 3), PLGVRGK (SEQ ID NO: 32), LGPKGQT (SEQ ID NO: 33), SGRSANAK (SEQ ID NO: 34), SGSKII (SEQ ID NO: 35), or GGLGPKGQTGGC (SEQ ID NO: 4).

9. The nanosensor of claim 8, wherein the cancer substrate is a metastatic cancer substrate, optionally wherein the metastatic cancer substrate is cleaved by one or more proteases selected from KLK2, KLK5, KLK12, KLK14, MMP3, MMP11, MMP13, PRSS3, ADAM12, and uPA, or optionally wherein the metastatic cancer substrate comprises the amino acid sequence set forth as GGGSGRSANAKGC (SEQ ID NO: 2), LGPKGQT (SEQ ID NO: 33), SGRSANAK (SEQ ID NO: 34), or GGLGPKGQTGGC (SEQ ID NO: 4).

10. The nanosensor of claim 8, wherein the cancer substrate is a non-metastatic cancer substrate, optionally wherein the non-metastatic cancer substrate comprises the amino acid sequence set forth as GPLGVRGKC (SEQ ID NO: 1), PLGVRGK (SEQ ID NO: 32), SGSKII (SEQ ID NO: 35), or GSGSKIIGGGC (SEQ ID NO: 3).

11. The nanosensor of claim 1, wherein the scaffold is linked to a single protease-specific substrate or wherein the scaffold is linked to 2 to 20 different protease-specific substrates.

12-13. (canceled)

14. The nanosensor of claim 1, wherein the detectable marker is a peptide, nucleic acid, small molecule, fluorophore (e.g., a fluorophore, or a fluorophore/quencher pair, such as a FRET pair), carbohydrate, particle, radiolabel, MRI-active compound, ligand encoded reporter, or isotope coded reporter molecule (iCORE).

15-21. (canceled)

22. A method comprising
detecting in a biological sample obtained from a subject that has been administered a prostate protease nanosensor or a composition of claim 1 one or more detectable markers that have been released from one or more prostate protease nanosensors when exposed to an enzyme present in the prostate of the subject.

23. The method of claim 22, wherein the biological sample is not a derived from the prostate of the subject, optionally wherein the sample is a urine sample, blood sample, or tissue sample.

24. (canceled)

25. The method of claim 22, wherein the subject has or is suspected of having prostate cancer.

26. The method of claim 22, further comprising the step of diagnosing the subject as having prostate cancer based upon the presence of the detectable markers in the biological sample, optionally wherein the subject is diagnosed as having indolent prostate cancer or aggressive prostate cancer.

27-35. (canceled)

36. A method for classifying cancer in a subject, the method comprising:
(i) detecting in a biological sample obtained from a subject that has been administered a prostate protease nanosensor or a composition of claim 1, wherein the biological sample is not derived from the prostate of the subject, one or more detectable markers that have been released from one or more prostate protease nanosensors when exposed to an enzyme present in the prostate of the subject; and
(ii) classifying the subject as having an indolent cancer or an aggressive cancer based on the identity of the detectable markers present in the biological sample, wherein the presence of the detectable markers in the biological sample is indicative of one or more cancer-associated enzymes being present in an active form within the prostate of the subject.

37. The method of claim 36, wherein the cancer is prostate cancer.

38. The method of claim 36, wherein the indolent cancer is non-metastatic cancer, optionally wherein the prostate cancer has a Gleason score of 6 or below.

39. The method of claim 38, wherein the cancer is classified as indolent based upon the presence of detectable markers released from a prostate protease nanosensor comprising a substrate including the amino acid sequence set forth as GPLGVRGKC (SEQ ID NO: 1), PLGVRGK (SEQ ID NO: 32), SGSKII (SEQ ID NO: 35), or GSGSKIIGGGC (SEQ ID NO: 3).

40. The method of claim 36, wherein the aggressive cancer is metastatic cancer, optionally wherein the prostate cancer has a Gleason score between 7 and 10.

41. The method of claim 40, wherein the cancer is classified as aggressive based upon the presence of detectable markers released from a prostate protease nanosensor having a substrate that is cleaved by one or more proteases selected from KLK2, KLK5, ADAM12, KLK12, KLK14, MMP3, MMP11, MMP13, PRSS3, and uPA, or a prostate protease nanosensor comprising a substrate having the amino acid sequence set forth as GGGSGRSANAKGC (SEQ ID NO: 2), LGPKGQT (SEQ ID NO: 33), SGRSANAK (SEQ ID NO: 34), or GGLGPKGQTGGC (SEQ ID NO: 4).

42-43. (canceled)

44. A method of treating prostate cancer in a subject, the method comprising administering a therapeutic agent for treatment of prostate cancer to or performing a therapeutic intervention on a subject who has been classified as having prostate cancer according to the method of claim 36.

45-46. (canceled) 6904133.1
